(19) United States
(12) Patent Application Publication
(10) Pub. No.: US 2025/0025439 A1
(43) Pub. Date: Jan. 23, 2025

(54) DERIVATIVES OF NIFUROXAZIDE FOR USE IN THE TREATMENT OF MITOCHONDRIAL DISORDERS AND NEURODEGENERATIVE DISEASES

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); UNIVERSITE COTE D'AZUR, Nice (FR); CENTRE HOSPITALIER RÉGIONAL, Nice (FR); ASSOCIATION FRANCAISE CONTRE LES MYOPATHIES (A.F.M.), Paris (FR)

(72) Inventors: Véronique PAQUIS-FLUCKLINGER, Nice Cedex 2 (FR); Stéphane AZOULAY, Nice Cedex (FR); Marc-Alexandre D'ELIA, Nice Cedex (FR); Baptiste ROPERT, Cologne (DE)

(21) Appl. No.: 18/708,662

(22) PCT Filed: Nov. 30, 2022
(86) PCT No.: PCT/EP2022/083829

§ 371 Date: May 9, 2024

(30) Foreign Application Priority Data
Nov. 30, 2021 (EP) 21306670.7

(51) Int. Cl. A61K 31/341; A61K 31/4178; A61K 31/422; A61K 31/5377; A61P 21/00; A61P 25/28

(52) U.S. Cl. CPC A61K 31/341; A61K 31/4178; A61K 31/422; A61K 31/5377; A61P 21/00; A61P 25/28

(57) ABSTRACT

Inventors have succeeded in identifying compounds comprising a nitrofuranyl moiety having the advantage of compensating mitochondrial dysfunction associated with both primary and secondary MICOS stability defects. These compounds are thus useful in preventing mitochondrial dysfunction associated with CHCHD10 mutations. Accordingly, the present invention relates to the use of nifuroxazide and its derivatives in the treatment of disorders associated with the disorganisation of the MICOS complex (MItochondrial contact site and Cristae Organizing System) and mitochondrial dysfunction such as mitochondrial disorders, in particular myopathy and cardiomyopathy, and neurodegenerative diseases, in particular motor neuron disease (including amyotrophic lateral sclerosis (ALS)) and frontotemporal dementia (FTD).

The present invention relates to the use of nifuroxazide and its derivatives in the treatment of disorders associated with the disorganisation of the MICOS complex (MItochondrial contact site and Cristae Organizing System) and mitochondrial dysfunction such as mitochondrial disorders, in particular myopathy and cardiomyopathy, and neurodegenerative diseases, in particular motor neuron disease (including amyotrophic lateral sclerosis (ALS)) and frontotemporal dementia (FTD).

### BACKGROUND OF THE INVENTION

Amyotrophic lateral sclerosis (ALS) is a devastating disease affecting upper and lower motor neurons leading to progressive failure of the neuromuscular system and death from respiratory failure within three to five years after symptoms begin (Parobkova et al., Diagnostics, 2021, 11, 509; Petrov et al., Neuroscience, 2017, 9, 68). Around 10% of ALS are classified as familial whereas the remaining 90% occur randomly and are considered as sporadic. Genetic research has revealed that ALS is not a single entity but rather a syndrome in which numerous causative genes have been identified. Twenty years after the discovery that 20% of familial ALS cases were associated with mutations in the superoxide dismutase 1 (SOD1) gene, the identification of the C9ORF72 expansion in 40% of familial ALS and 25% of familial Frontotemporal Dementia (FTD) provided strong evidence that ALS and FTD are manifestations of a clinicopathological spectrum (DeJesus-Hernandez et al., Neuron, 2011, 72, 245-256; Renton et al., Neuron, 2011, 72, 257-268; Rosen et al., Nature, 1993, 362, 59-62). Indeed, it has been estimated that 15% of FTD patients develop features of ALS and, up to 50% of ALS patients show frontal lobe dysfunction. Among a growing number of genes involved in ALS and FTD clinical spectrum, mutations in three genes account for the majority of cases. They correspond to two genes encoding RNA/DNA binding proteins, TDP-43 (TARDBP or TAR-DNA-binding protein-43) (Neumann et al., Science, 2006, 314, 130-133) and FUS-TLS (fused in sarcoma/translocation in liposarcoma or FUS) (Kwiatkowski et al., Science, 2009, 323, 1205-1208; Vance et al., Science, 2009, 323, 1208-1211), and to the GGGGCC hexanucleotide expansion in the C9ORF72 gene (DeJesus-Hernandez et al., Neuron, 2011, 72, 245-256; Renton et al., Neuron, 2011, 72, 257-268).

Several other genes have been successively identified in FTD-ALS spectrum (OPTN, VCP, UBQLN2, STSQM1, PFN1 . . . ) (Parobkova et al., Diagnostics, 2021, 11, 509; Renton et al., Nat. Neurosci., 2014, 17, 17-23) showing that RNA dysregulation, impaired protein homeostasis, oxidative damage, defective neuronal transport, neuroinflammation, excitotoxicity . . . are involved in this disease (Ferraiuolo et al., Nat. Rev. Neurol., 2011, 7, 616-630; Parobkova et al., Diagnostics, 2021, 11, 509).

Among all factors involved in ALS pathogenesis, mitochondrial dysfunction has always been recognized as a major player because abnormal mitochondrial structure, respiratory chain (RC) deficiency, increased oxidative stress or induction of apoptosis have been found in ALS patients and models (Cozzolino et al., Front. Cell. Neurosci., 2015, 9:31). Secondary mitochondrial dysfunction has been described in ALS but also in many neurodegenerative diseases. However, a causative role of mitochondria had always been debated. The identification of the gene encoding the Coiled-Coil-Helix-Coiled-Coil-Helix Domain Containing protein 10 (CHCHD10) was the first genetic evidence demonstrating that a mitochondrial defect can trigger motor neuron disease (MND). A large family with a late-onset phenotype including a mitochondrial myopathy with MND and cognitive decline resembling FTD has been reported and a heterozygous variant (p.Ser59Leu) in CHCHD10 encoding a mitochondrial protein enriched at cristae junctions has been identified (Bannwarth et al., Brain, 2014, 137, 2329-2345). Rapidly, it has been showed that CHCHD10 is involved in a huge clinical spectrum including early-onset mitochondrial myopathy (Ajroud-Driss et al., Neurogenetics, 2015, 16, 1-9), late-onset spinal motor neuropathy (SMAJ) (Penttila et al., Ann. Neurol., 2015, 77, 163-172), autosomal dominant cardiomyopathy (Liu et al., Hum. Mol. Gen. 2020, 29, 1547-1567; Salmon et al., The Lancet, 1971, 298, 290-293), ALS, FTD (Cozzolino et al., Front. Cell. Neurosci., 2015, 9:31; Parobkova et al., Diagnostics, 2021, 11, 509), FTD-ALS (Chaussenot et al., Neurobiology of Aging, 2014, 35, 2884.e1-2884.e4) or Charcot-Marie-Tooth disease type 2 (CMT2) (Auranen et al., Neurol. Genet., 2015, 1, e1).

Fibroblasts of patients carrying the p.Ser59Leu variant (CHCHD10^{S59L/+}) display both a fragmented mitochondrial network and abnormal mitochondrial morphology with loss of cristae (Bannwarth et al., Brain, 2014, 137, 2329-2345). Mitofilin/MIC60, a protein enriched at mitochondrial cristae junctions, is a central component of mitochondrial contact site and cristae organizing system (MICOS) complex (Pfanner et al., J. Cell. Biol., 2014, 204, 1083-1086). Depletion of Mitofilin in human cells or deletion in yeast leads to abnormal cristae structures with a massive loss of cristae junctions. Destabilization of MICOS also correlates with concomitant loss of cristae junctions, which indicates that the integrity of MICOS is required for the formation and/or maintenance of these structures (Friedman et al., ELife, 2015, 4, e07739). CHCHD10 is a partner of Mitofilin and is involved in MICOS stability (Genin et al., EMBO Mol. Med., 2016, 8, 58-72; Liu et al., FASEB J., 2020, 34, 8493-8509). In patient fibroblasts, the expression of the CHCHD10^{S59L} mutant allele leads to MICOS complex disassembly that is at least in part involved in the loss of mitochondrial cristae (Genin et al., EMBO Mol. Med., 2016, 8, 58-72). This suggests that the maintenance of MICOS integrity could be a target to prevent mitochondrial dysfunction associated with CHCHD10 mutations.

Hence, there is a need for compounds having the ability to prevent mitochondrial dysfunction associated with CHCHD10 mutations, and that may be of therapeutic value in the treatment of diseases associated with mitochondrial dysfunction such as mitochondrial disorders, in particular myopathy and cardiomyopathy, and neurodegenerative diseases, in particular motor neuron disease such as ALS, and FTD.

### SUMMARY OF THE INVENTION

The inventors have now succeeded in identifying compounds comprising a nitrofuranyl moiety having the advantage of compensating mitochondrial dysfunction associated with both primary and secondary MICOS stability defects. These compounds are thus useful in preventing mitochondrial dysfunction associated with CHCHD10 mutations.

The invention therefore relates to compounds of general Formula I, their pharmaceutically acceptable salts and solvates, for use in the treatment of diseases associated with mitochondrial dysfunction, in particular mitochondrial disorders and neurodegenerative diseases.

In a general aspect, the invention provides a compound of general Formula I:

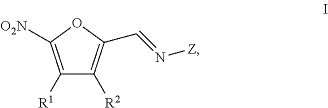

a pharmaceutically acceptable salt or a solvate thereof,
wherein
R¹ and R² are independently selected from H, C1-C6-alkyl, C3-C6-cycloalkyl and halogens,
Z is selected from

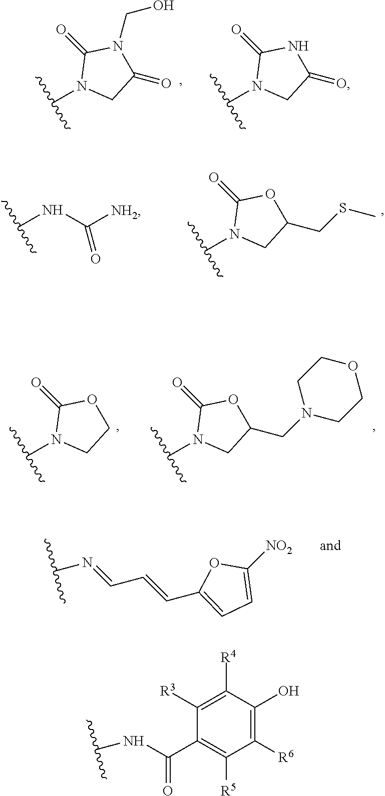

wherein R³, R⁴, R⁵ and R⁶ are independently selected from H, C1-C6-alkyl, C3-C6-cycloalkyl, OH and halogens,
   for use in treating diseases associated with mitochondrial dysfunction.

In a second aspect, the invention provides a pharmaceutical composition comprising a compound Formula I:

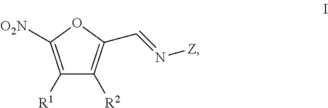

or a pharmaceutically acceptable salt or solvate thereof and at least one pharmaceutically acceptable excipient,
wherein
R¹ and R² are independently selected from H, C1-C6-alkyl, C3-C6-cycloalkyl and halogens,
Z is selected from

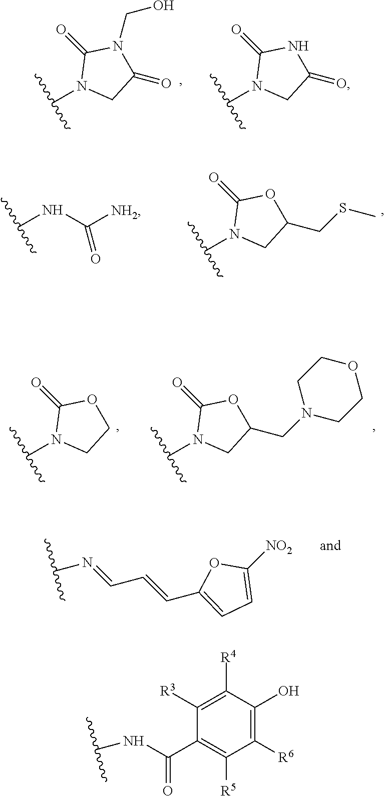

wherein R³, R⁴, R⁵ and R⁶ are independently selected from H, C1-C6-alkyl, C3-C6-cycloalkyl, OH and halogens,
   for use in treating diseases associated with mitochondrial dysfunction.

### DETAILED DESCRIPTION OF THE INVENTION

Unexpectedly, the inventors have discovered that the compound of Formula I is able to compensate mitochondrial dysfunction associated with both primary and secondary MICOS stability defects.

The compounds of Formula I can thus be used for treating disorders associated with MICOS instability and mitochondrial dysfunction, such as mitochondrial disorders, in particular myopathy and cardiomyopathy, and neurodegenerative diseases, in particular motor neuron disease, more particularly ALS, and FTD.

The invention thus relates to a compound of Formula I:

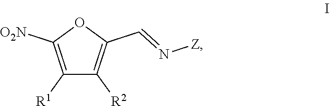

a pharmaceutically acceptable salt or a solvate thereof,
wherein
R¹ and R² are independently selected from H, C1-C6-alkyl, C3-C6-cycloalkyl and halogens,
Z is selected from

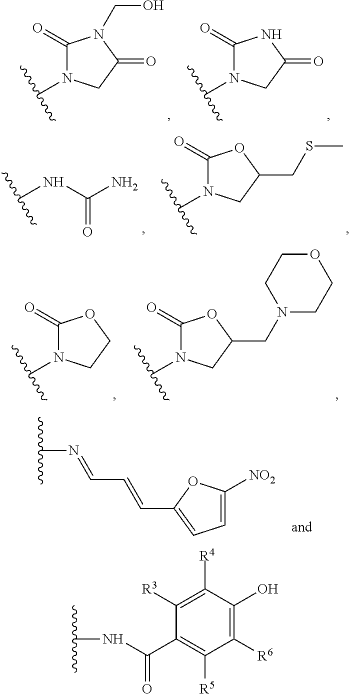

wherein R³, R⁴, R⁵ and R⁶ are independently selected from H, C1-C6-alkyl, C3-C6-cycloalkyl, OH and halogens,
   for use in treating diseases associated with mitochondrial dysfunction, in particular mitochondrial disorders and neurodegerative diseases.

Halogens include a fluorine atom, an iodine atom, a chlorine atom and a bromine atom.

C1-C6-alkyl include hexyl, in particular n-hexyl, neohexyl, isohexyl, sec-hexyl or tert-hexyl; pentyl, in particular n-pentyl, neopentyl, isopentyl, sec-pentyl or tert-pentyl; butyl, in particular n-butyl, isobutyl, sec-butyl or tert-butyl; propyl, in particular n-propyl or isopropyl; ethyl or methyl.

C3-C6-cycloalkyl include cyclopropyl, cyclobutyl, cyclopentyl and cyclohexyl.

In one embodiment, particular compounds for use according to the invention are compounds of formula I, or pharmaceutically acceptable salts or solvates thereof, wherein one or more of R¹, R², R³, R⁴, R⁵, R⁶ and Z are defined as follows:
R¹ and R² are independently selected from H, C1-C6-alkyl, such as hexyl, in particular n-hexyl, neohexyl, isohexyl, sec-hexyl or tert-hexyl; pentyl, in particular n-pentyl, neopentyl, isopentyl, sec-pentyl or tert-pentyl; butyl, in particular n-butyl, isobutyl, sec-butyl or tert-butyl; propyl, in particular n-propyl or isopropyl; ethyl or methyl; and halogens; particularly, R¹ and R² are independently selected from H and C1-C4-alkyl, such as butyl, in particular n-butyl, isobutyl, sec-butyl or tert-butyl; propyl, in particular n-propyl or isopropyl; ethyl or methyl; more particularly, R¹ and R² are independently selected from H and C1-C2-alkyl, such as ethyl or methyl; still more particularly, R¹ and R² are H;
Z is selected from

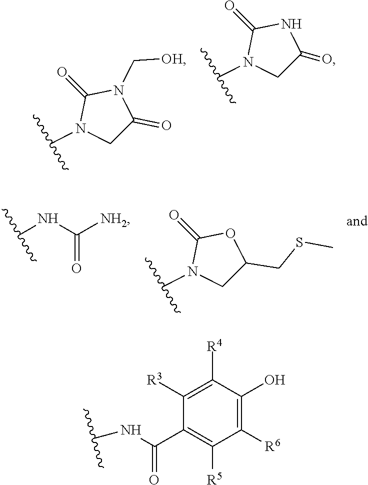

wherein R³, R⁴, R⁵ and R⁶ are independently selected from H, C1-C6-alkyl, C3-C6-cycloalkyl, OH and halogens; in particular R³, R⁴, R⁵ and R⁶ are independently selected from H, C1-C6-alkyl, OH and halogens, more particularly R³, R⁴, R⁵ and R⁶ are independently selected from H and C1-C4-alkyl; still more particularly R³, R⁴, R⁵ and R⁶ are independently selected from H and C1-C2-alkyl; even more particularly R³, R⁴, R⁵ and R⁶ are H; in particular, Z is selected from

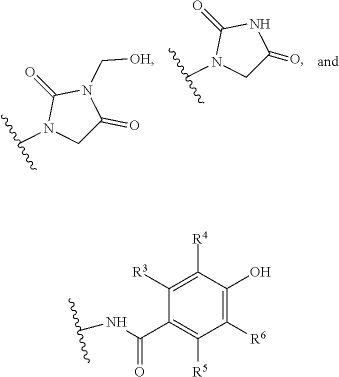

wherein R³, R⁴, R⁵ and R⁶ are independently selected from H, C1-C6-alkyl, C3-C6-cycloalkyl, OH and halogens; in particular R³, R⁴, R⁵ and R⁶ are independently selected from H, C1-C6-alkyl, OH and halogens, more particularly R³, R⁴, R⁵ and R⁶ are independently selected from H and C1-C4-alkyl; still more particularly R³, R⁴, R⁵ and R⁶ are independently selected from H and C1-C2-alkyl; even more particularly R³, R⁴, R⁵ and R⁶ are H; more particularly, Z is selected from and

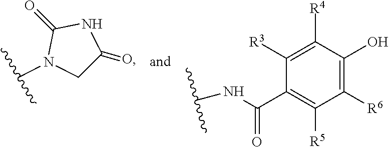

wherein R³, R⁴, R⁵ and R⁶ are independently selected from H, C1-C6-alkyl, C3-C6-cycloalkyl, OH and halogens; in particular R³, R⁴, R⁵ and R⁶ are independently selected from H, C1-C6-alkyl, OH and halogens, more particularly R³, R⁴, R⁵ and R⁶ are independently selected from H and C1-C4-alkyl; still more particularly R³, R⁴, R⁵ and R⁶ are independently selected from H and C1-C2-alkyl; even more particularly R³, R⁴, R⁵ and R⁶ are H; still more particularly, Z is and

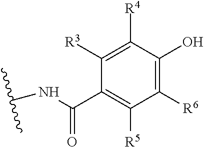

wherein R³, R⁴, R⁵ and R⁶ are independently selected from H, C1-C6-alkyl, C3-C6-cycloalkyl, OH and halogens; in particular R³, R⁴, R⁵ and R⁶ are independently selected from H, C1-C6-alkyl, OH and halogens, more particularly R³, R⁴, R⁵ and R⁶ are independently selected from H and C1-C4-alkyl; still more particularly R³, R⁴, R⁵ and R⁶ are independently selected from H and C1-C2-alkyl; even more particularly R³, R⁴, R⁵ and R⁶ are H; preferably Z is selected from

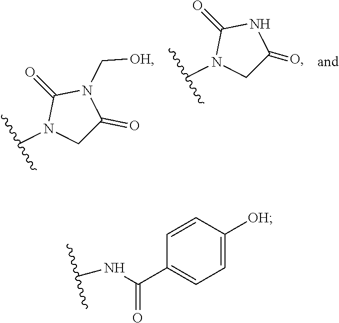 [SMILES: *N1CC(=O)N(CO)C1=O ; *N1CC(=O)NC1=O ; *NC(=O)c1ccc(O)cc1]

more preferably Z is

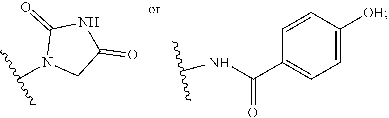 [SMILES: *N1CC(=O)NC1=O ; *NC(=O)c1ccc(O)cc1]

still more preferably Z is

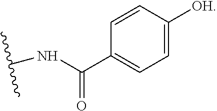 [SMILES: *NC(=O)c1ccc(O)cc1]

In one embodiment, the compounds for use according to the invention are compounds of formula I, or pharmaceutically acceptable salts or solvates thereof, wherein Z is

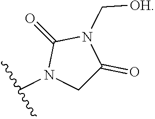 [SMILES: *N1CC(=O)N(CO)C1=O]

In one embodiment, the compounds for use according to the invention are compounds of formula I, or pharmaceutically acceptable salts or solvates thereof, wherein Z is

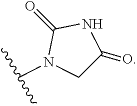 [SMILES: *N1CC(=O)NC1=O]

In one embodiment, the compounds for use according to the invention are compounds of formula I, or pharmaceutically acceptable salts or solvates thereof, wherein Z is

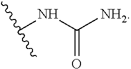 [SMILES: *NC(N)=O]

In one embodiment, the compounds for use according to the invention are compounds of formula I, or pharmaceutically acceptable salts or solvates thereof, wherein Z is

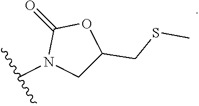 [SMILES: *N1CC(CSC)OC1=O]

In one embodiment, the compounds for use according to the invention are compounds of formula I, or pharmaceutically acceptable salts or solvates thereof, wherein Z is

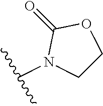 [SMILES: *N1CCOC1=O]

In one embodiment, the compounds for use according to the invention are compounds of formula I, or pharmaceutically acceptable salts or solvates thereof, wherein Z is

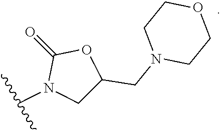 [SMILES: *N1CC(CN2CCOCC2)OC1=O]

In one embodiment, the compounds for use according to the invention are compounds of formula I, or pharmaceutically acceptable salts or solvates thereof, wherein Z is

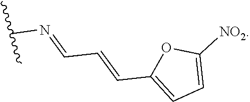 [SMILES: */N=C/C=C/c1ccc([N+](=O)[O-])o1]

In one embodiment, the compounds for use according to the invention are compounds of formula I, or pharmaceutically acceptable salts or solvates thereof, wherein Z is

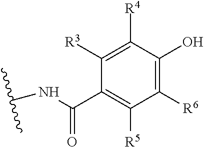

wherein R³, R⁴, R⁵ and R⁶ are independently selected from H, C1-C6-alkyl, C3-C6-cycloalkyl, OH and halogens; in particular R³, R⁴, R⁵ and R⁶ are independently selected from H, C1-C6-alkyl, OH and halogens, more particularly R³, R⁴, R⁵ and R⁶ are independently selected from H and C1-C4-alkyl; still more particularly R³, R⁴, R⁵ and R⁶ are independently selected from H and C1-C2-alkyl; even more particularly R³, R⁴, R⁵ and R⁶ are H.

In one embodiment, particularly preferred compounds for use according to the invention are those listed in Table 1 hereafter:

TABLE 1

| Compound | Structure | Name |
|---|---|---|
| 1 | 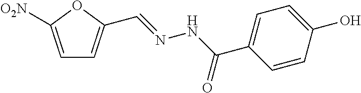 [SMILES: O=C(N/N=C/c1ccc([N+](=O)[O-])o1)c1ccc(O)cc1] | (E)-4-hydroxy-N′-((5-nitrofuran-2- yl)methylene)benzohydrazide (Nifuroxazide) |
| 2 | 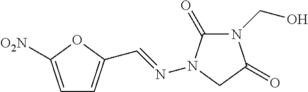 [SMILES: O=C1CN(/N=C/c2ccc([N+](=O)[O-])o2)C(=O)N1CO] | (E)-3-(hydroxymethyl)-1-(((5- nitrofuran-2- yl)methylene)amino)imidazolidine-2,4- dione (Nifurtoinol) |
| 3 | 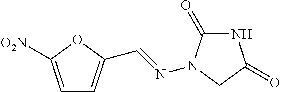 [SMILES: O=C1CN(/N=C/c2ccc([N+](=O)[O-])o2)C(=O)N1] | (E)-1-(((5-nitrofuran-2- yl)methylene)amino)imidazolidine-2,4- dione (Nitrofurantoin) |
| 4 | 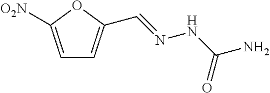 [SMILES: NC(=O)N/N=C/c1ccc([N+](=O)[O-])o1] | (E)-2-((5-nitrofuran-2- yl)methylene)hydrazine-1-carboxamide (Nitrofurazone) |
| 5 | 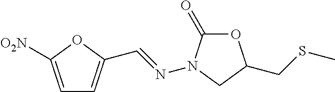 [SMILES: CSCC1CN(/N=C/c2ccc([N+](=O)[O-])o2)C(=O)O1] | (E)-5-((methylthio)methyl)-3-(((5- nitrofuran-2- yl)methylene)amino)oxazolidin-2-one (Nifuratel) |
| 6 | 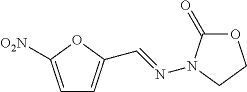 [SMILES: O=C1OCCN1/N=C/c1ccc([N+](=O)[O-])o1] | (E)-3-(((5-nitrofuran-2- yl)methylene)amino)oxazolidin-2-one (Furazolidone) |
| 7 | 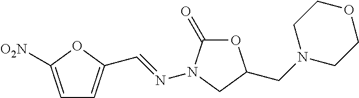 [SMILES: O=C1OC(CN2CCOCC2)CN1/N=C/c1ccc([N+](=O)[O-])o1] | (E)-5-(morpholinomethyl)-3-(((5- nitrofuran-2- yl)methylene)amino)oxazolidin-2-one (Furaltadone) |
| 8 | 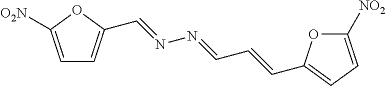 [SMILES: O=[N+]([O-])c1ccc(/C=C/C=N/N=C/c2ccc([N+](=O)[O-])o2)o1] | (1E,2E)-1-((E)-3-(5-nitrofuran-2- yl)allylidene)-2-((5-nitrofuran-2- yl)methylene)hydrazine (Furazidine) |

More particularly, the compound for use according to the invention is selected from compound 1 (Nifuroxazide), compound 2 (Nifurtoinol) and compound 3 (Nitrofurantoin).

Still more particularly, the compound for use according to the invention is selected from compound 1 (Nifuroxazide) and compound 3 (Nitrofurantoin).

Even more particularly, the compound for use according to the invention is compound 1 (Nifuroxazide).

The compounds of the invention can be prepared by different ways with reactions known by the person skilled in the art or are commercially available.

The compounds of Formula I are indeed capable of compensating mitochondrial dysfunction associated with both primary and secondary MICOS stability defects. They further have the advantage of preventing mitochondrial dysfunction associated with CHCHD10 mutations.

Accordingly, in a particularly preferred embodiment, the invention relates to the use of compounds of Formula I or pharmaceutically acceptable salts or solvates thereof, for the treatment of diseases associated with MICOS disassembly and mitochondrial dysfunction, in particular diseases associated with mitochondrial dysfunction.

Diseases associated with MICOS disassembly and mitochondrial dysfunction, in particular diseases associated with mitochondrial dysfunction, include mitochondrial disorders, in particular myopathy and cardiomyopathy, and neurodegenerative diseases, in particular motor neuron disease, more particularly amyotrophic lateral sclerosis, and frontotemporal dementia.

The invention thus also relates to a compound of the present invention, in particularly a compound of Formula I, more particularly a compound of Table 1 above, or a pharmaceutically acceptable salt or solvate thereof, for use in treating diseases associated with mitochondrial dysfunction.

In one embodiment, the diseases associated with mitochondrial dysfunction are selected from mitochondrial disorders and neurodegenerative diseases.

In one embodiment, the diseases associated with mitochondrial dysfunction are selected from mitochondrial disorders.

In one embodiment, the mitochondrial disorders are selected from myopathy and cardiomyopathy.

In one embodiment, the mitochondrial disorder is myopathy.

In one embodiment, the mitochondrial disorder is cardiomyopathy.

In one embodiment, the diseases associated with mitochondrial dysfunction are selected from neurodegenerative diseases.

In one embodiment, the neurodegenerative diseases are selected from motor neuron disease, in particular amyotrophic lateral sclerosis, and frontotemporal dementia. More particularly, the neurodegenerative diseases are selected from amyotrophic lateral sclerosis and frontotemporal dementia. Still more particularly, the neurodegenerative disease is amyotrophic lateral sclerosis.

In one embodiment, the motor neuron disease is amyotrophic lateral sclerosis.

In one embodiment, the neurodegenerative disease is amyotrophic lateral sclerosis.

In one embodiment, the neurodegenerative disease is frontotemporal dementia.

In other terms, the invention also relates to a method of treating diseases associated with mitochondrial dysfunction, in particular mitochondrial disorders and neurodegenerative diseases, comprising the administration of a therapeutically effective amount of a compound of Formula I, in particular a compound of Table 1 above, or a pharmaceutically acceptable salt or solvate thereof, to a patient in need of such treatment. Preferably the patient is a warm-blooded animal, more preferably a human.

In one embodiment, the invention relates to a method of treating mitochondrial disorders, comprising the administration of a therapeutically effective amount of a compound of Formula I, in particular a compound of Table 1 above, or a pharmaceutically acceptable salt or solvate thereof, to a patient in need of such treatment.

In one embodiment, the invention relates to a method of treating neurodegenerative diseases, comprising the administration of a therapeutically effective amount of a compound of Formula I, in particular a compound of Table 1 above, or a pharmaceutically acceptable salt or solvate thereof, to a patient in need of such treatment.

The invention further provides the use of a compound of Formula I, in particular a compound of Table 1 above, or a pharmaceutically acceptable salt or solvates thereof, for the manufacture of a medicament for use in treating diseases associated with mitochondrial dysfunction, in particular mitochondrial disorders and neurodegenerative diseases. Preferably the patient is a warm-blooded animal, more preferably a human.

In one embodiment, the invention provides the use of a compound of Formula I, in particular a compound of Table 1 above, or a pharmaceutically acceptable salt or solvates thereof, for the manufacture of a medicament for use in treating mitochondrial disorders.

In one embodiment, the invention provides the use of a compound of Formula I, in particular a compound of Table 1 above, or a pharmaceutically acceptable salt or solvates thereof, for the manufacture of a medicament for use in treating neurodegenerative diseases.

According to a further feature of the present invention, there is provided the use of a compound of the present invention, or a pharmaceutically acceptable salt or solvate thereof, for preventing mitochondrial dysfunction associated with CHCHD10 mutations, in a patient in need of such treatment, comprising administering to said patient an effective amount of a compound of the present invention, or a pharmaceutically acceptable salt or solvate thereof. In other terms, the invention also provides a method for preventing mitochondrial dysfunction associated with CHCHD10 mutations, in a patient in need of such treatment, which comprises the step of administering to said patient an effective amount of a compound of the present invention, or a pharmaceutically acceptable salt or solvate thereof. Preferably, the patient is a warm blooded animal, and even more preferably a human.

According to the present invention, the compound of the invention may be administered as a pharmaceutical formulation in a therapeutically effective amount by any of the accepted modes of administration, in particular by intravenous, transcutaneous or oral route, more particularly by intravenous or transcutaneous route.

Therapeutically effective amount ranges are typically from 0.1 to 50 000 μg/kg of body weight daily, preferably from 1 000 to 40 000 μg/kg of body weight daily, depending upon numerous factors such as the severity of the disease to be treated, the age and relative health of the subject, the potency of the compound, the route and the form of administration, the indication towards which the administration is directed, and the preferences and experience of the medical practitioner involved. One of ordinary skill in the art of treating such diseases will be able in reliance upon personal knowledge, to ascertain a therapeutically effective amount of the compound for use of the present invention for a given mitochondrial disorder or neurodegenerative disease.

The methods of treatment and pharmaceutical compositions of the present invention may employ the compounds of the invention or their pharmaceutical acceptable salts or solvates thereof in the form of monotherapy, but said methods and compositions may also be used in the form of multiple therapy in which one or more compounds of the invention or their pharmaceutically acceptable salts or solvates are co-administered in combination with one or more other therapeutic agents.

The invention also provides pharmaceutical compositions comprising a compound of formula I, in particular a compound of Table 1 above, or a pharmaceutically acceptable salt or solvate thereof, and at least one pharmaceutically acceptable carrier, diluent, excipient and/or adjuvant, for use in the treatment of diseases associated with mitochondrial dysfunction, in particular mitochondrial disorders and neurodegenerative diseases. As indicated above, the invention also covers pharmaceutical compositions which contain, in addition to a compound of the present invention, a pharmaceutically acceptable salt or solvate thereof as active ingredient, additional therapeutic agents and/or active ingredients.

In one embodiment, the diseases associated with mitochondrial dysfunction are selected from mitochondrial disorders and neurodegenerative diseases.

In one embodiment, the diseases associated with mitochondrial dysfunction are selected from mitochondrial disorders.

In one embodiment, the mitochondrial disorders are selected from myopathy and cardiomyopathy.

In one embodiment, the mitochondrial disorder is myopathy.

In one embodiment, the mitochondrial disorder is cardiomyopathy.

In one embodiment, the diseases associated with mitochondrial dysfunction are selected from neurodegenerative diseases.

In one embodiment, the neurodegenerative diseases are selected from motor neuron disease, in particular amyotrophic lateral sclerosis, and frontotemporal dementia. More particularly, the neurodegenerative diseases are selected from amyotrophic lateral sclerosis and frontotemporal dementia. Still more particularly, the neurodegenerative disease is amyotrophic lateral sclerosis.

In one embodiment, the motor neuron disease is amyotrophic lateral sclerosis.

In one embodiment, the neurodegenerative disease is amyotrophic lateral sclerosis.

In one embodiment, the neurodegenerative disease is frontotemporal dementia.

The invention also provides a compound of the invention, or a pharmaceutically acceptable salt or solvate thereof, for use in a therapeutic treatment in humans or animals.

Generally, for pharmaceutical use, the compounds for use of the invention may be formulated as a pharmaceutical preparation comprising at least one compound for use of the invention and at least one pharmaceutically acceptable carrier, diluent, excipient and/or adjuvant, and optionally one or more further pharmaceutically active compounds.

By means of non-limiting examples, such a formulation may be in a form suitable for oral administration, for transcutaneous administration, e.g. parenteral administration (such as by intravenous, intramuscular, intracerebral or subcutaneous injection or intravenous infusion), or by an implant, for topical administration (including ocular), for administration by inhalation, by a skin patch, by an implant, by a suppository, etc. Preferably, the formulation may be in a form suitable for oral administration, for parenteral administration (such as by intravenous, intramuscular, intracerebral or subcutaneous injection or intravenous infusion). Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

For example, the compound for use of the invention or a pharmaceutical composition comprising a compound of the invention can be administered orally in the form of tablets, coated tablets, pills, capsules, soft gelatin capsules, oral powders, granules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, a disintegrant such as starch (preferably corn, potato or tapioca starch), sodium starch glycollate, croscarmellose sodium and certain complex silicates, a binder such as polyvinylpyrrolidone, hydroxypropylmethylcellulose (HPMC), hydroxypropylcellulose (HPC), sucrose, gelatin and acacia, a lubricant such as magnesium stearate, stearic acid, glyceryl behenate. Solid compositions of a similar type may also be employed as fillers in hard gelatin capsules. Preferred excipients in this regard include lactose, saccharose, sorbitol, mannitol, potato starch, corn starch, amylopectin, cellulose derivatives or gelatin. Hard gelatin capsules may contain granules of the compound of the invention.

Soft gelatin capsules may be prepared with capsules containing the compound of the invention, vegetable oil, waxes, fat, or other suitable vehicle for soft gelatin capsules. As an example, the acceptable vehicle can be an oleaginous vehicle, such as a long chain triglyceride vegetable oil (e.g. corn oil).

Dispersible powders and granules suitable for preparation of an aqueous suspension by the addition of water may contain the active ingredient in a mixture with dispersing agents, wetting agents, and suspending agents and one or more preservatives. Additional excipients, for example sweetening, flavouring and colouring agents, may also be present. These compositions may be preserved by the addition of an anti-oxidant such as ascorbic acid.

Liquid dosage forms for oral administration may include pharmaceutically acceptable, solutions, emulsions, suspensions, syrups, and elixirs containing inert diluents commonly used in the art, such as water or an oleaginous vehicle. Liquid dosage form may be presented as a dry product for constitution with water or other suitable vehicle before use. Such compositions may also comprise adjuvants, such as wetting agents, emulsifying and suspending agents, complexing agents such as 2-hydroxypropyl-beta-cyclodextrin, sulfobutylether-beta-cylodextrin, and sweetening, flavouring, perfuming agents, colouring matter or dyes with diluents such as water, ethanol, propylene glycol and glycerin, and combinations thereof. These compositions may be preserved by the addition of an anti-oxidant such as butylated hydroxyanisol or alpha-tocopherol.

Finely divided powder of the compound for use of the invention may be prepared for example by micronisation or by processes known in the art. The compound of the invention may be milled using known milling procedures such as wet milling to obtain a particle size appropriate for tablet formation and for other formulation types.

If the compound for use of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent; and/or by using infusion techniques.

The compound for use of the invention can be administered via the parenteral route with a readily available or a depot-type formulation.

The pharmaceutical compositions for the parenteral administration of a readily available formulation may be in the form of a sterile injectable aqueous or oleagenous solution or suspension in a non-toxic parenterally-acceptable diluent or solvent and may contain formulatory agents such as suspending, stabilising dispersing, wetting and/or complexing agents such as cyclodextrin e.g. 2-hydroxypropyl-beta-cyclodextrin, sulfobutylether-beta-cylodextrin.

The depot-type formulation for the parenteral administration may be prepared by conventional techniques with pharmaceutically acceptable excipient including without being limited to, biocompatible and biodegradable polymers (e.g. poly(O-caprolactone), poly(ethylene oxide), poly(glycolic acid), poly[(lactic acid)-co-(glycolic acid) . . . )], poly(lactic acid) . . . ), non-biodegradable polymers (e.g. ethylene vinylacetate copolymer, polyurethane, polyester(amide), polyvinyl chloride . . . ) aqueous and non-aqueous vehicles (e.g. water, sesame oil, cottonseed oil, soybean oil, castor oil, almond oil, oily esters, ethyl alcohol or fractionated vegetable oils, propylene glycol, DMSO, THF, 2-pyrrolidone, N-methylpyrrolidinone, N-vinylpyrrolidinone . . . ).

Alternatively, the active ingredient may be in dry form such as a powder, crystalline or freeze-dried solid for constitution with a suitable vehicle. The preparation of suitable parenteral formulations under sterile conditions is readily accomplished by standard pharmaceutical techniques well known to those skilled in the art.

The present invention also relates to a compound of general Formula I:

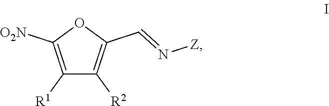

a pharmaceutically acceptable salt or a solvate thereof,
wherein
R¹ and R² are independently selected from H, C1-C6-alkyl, C3-C6-cycloalkyl and halogens,
Z is selected from

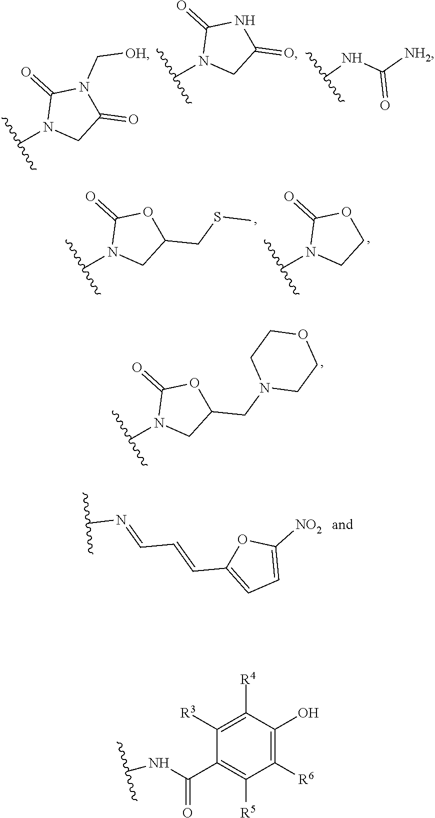

wherein R³, R⁴, R⁵ and R⁶ are independently selected from H, C1-C6-alkyl, C3-C6-cycloalkyl, OH and halogens,
   with the proviso that R¹ and R² are not both H and that R³, R⁴, R⁵ and R⁶ are not all H.

In one embodiment, the compound of Formula I is none of the following:
(E)-4-hydroxy-N′-((5-nitrofuran-2-yl)methylene)benzohydrazide);
(E)-3-(hydroxymethyl)-1-(((5-nitrofuran-2-yl)methylene)amino)imidazolidine-2,4-dione;
(E)-1-(((5-nitrofuran-2-yl)methylene)amino)imidazolidine-2,4-dione;
(E)-2-((5-nitrofuran-2-yl)methylene)hydrazine-1-carboxamide;
(E)-5-((methylthio)methyl)-3-(((5-nitrofuran-2-yl)methylene)amino)oxazolidin-2-one;
(E)-3-(((5-nitrofuran-2-yl)methylene)amino)oxazolidin-2-one;
(E)-5-(morpholinomethyl)-3-(((5-nitrofuran-2-yl)methylene)amino)oxazolidin-2-one; and
(1E,2E)-1-((E)-3-(5-nitrofuran-2-yl)allylidene)-2-((5-nitrofuran-2-yl)methylene)hydrazine.

In one embodiment, particular compounds according to the invention are compounds of formula I, or pharmaceutically acceptable salts or solvates thereof, wherein one or more of R¹, R², R³, R⁴, R⁵, R⁶ and Z are defined as follows:
R¹ and R² are independently selected from H, C1-C6-alkyl, such as hexyl, in particular n-hexyl, neohexyl, isohexyl, sec-hexyl or tert-hexyl; pentyl, in particular n-pentyl, neopentyl, isopentyl, sec-pentyl or tert-pentyl; butyl, in particular n-butyl, isobutyl, sec-butyl or tert-butyl; propyl, in particular n-propyl or isopropyl; ethyl or methyl; and halogens; particularly, R¹ and R² are independently selected from H and C1-C4-alkyl, such as butyl, in particular n-butyl, isobutyl, sec-butyl or tert-butyl; propyl, in particular n-propyl or isopropyl; ethyl or methyl; more particularly, R¹ and R² are independently selected from H and C1-C2-alkyl, such as ethyl or methyl;
Z is selected from

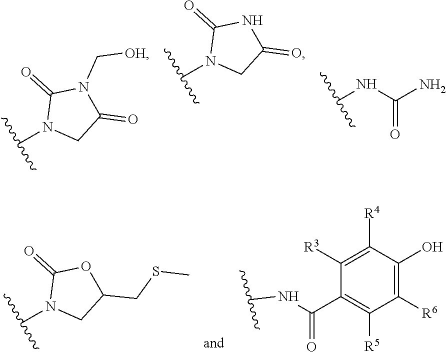

wherein R³, R⁴, R⁵ and R⁶ are independently selected from H, C1-C6-alkyl, C3-C6-cycloalkyl, OH and halogens; in particular R³, R⁴, R⁵ and R⁶ are independently selected from H, C1-C6-alkyl, OH and halogens, more particularly R³, R⁴, R⁵ and R⁶ are independently selected from H and C1-C4-alkyl; still more particularly R³, R⁴, R⁵ and R⁶ are independently selected from H and C1-C2-alkyl; in particular, Z is selected from

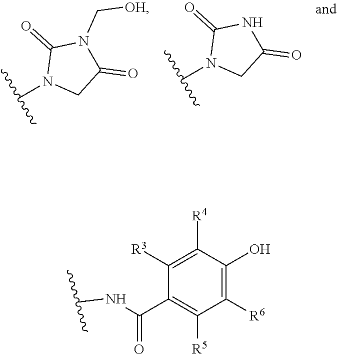

wherein R³, R⁴, R⁵ and R⁶ are independently selected from H, C1-C6-alkyl, C3-C6-cycloalkyl, OH and halogens; in particular R³, R⁴, R⁵ and R⁶ are independently selected from H, C1-C6-alkyl, OH and halogens, more particularly R³, R⁴, R⁵ and R⁶ are independently selected from H and C1-C4-alkyl; still more particularly R³, R⁴, R⁵ and R⁶ are independently selected from H and C1-C2-alkyl; more particularly, Z is selected from

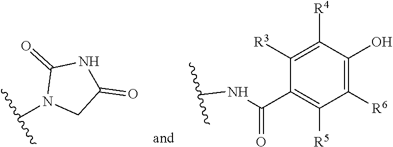

wherein R³, R⁴, R⁵ and R⁶ are independently selected from H, C1-C6-alkyl, C3-C6-cycloalkyl, OH and halogens; in particular R³, R⁴, R⁵ and R⁶ are independently selected from H, C1-C6-alkyl, OH and halogens, more particularly R³, R⁴, R⁵ and R⁶ are independently selected from H and C1-C4-alkyl; still more particularly R³, R⁴, R⁵ and R⁶ are independently selected from H and C1-C2-alkyl; still more particularly, Z is and

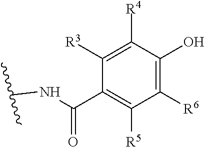

wherein R³, R⁴, R⁵ and R⁶ are independently selected from H, C1-C6-alkyl, C3-C6-cycloalkyl, OH and halogens; in particular R³, R⁴, R⁵ and R⁶ are independently selected from H, C1-C6-alkyl, OH and halogens, more particularly R³, R⁴, R⁵ and R⁶ are independently selected from H and C1-C4-alkyl; still more particularly R³, R⁴, R⁵ and R⁶ are independently selected from H and C1-C2-alkyl.

### Definitions

The definitions and explanations below are for the terms as used throughout the entire application, including both the specification and the claims.

Unless otherwise stated, any reference to compounds of the invention herein, means the compounds as such as well as their pharmaceutically acceptable salts and solvates.

When describing the compounds for use of the invention, the terms used are to be construed in accordance with the following definitions, unless indicated otherwise.

The term “halo” or “halogen” refers to the atoms of the group 17 of the periodic table (halogens) and includes in particular fluorine, chlorine, bromine and iodine atom.

The term “alkyl” by itself or as part of another substituent refers to a hydrocarbyl group of Formula CₙH₂ₙ₊₁ wherein n is a number greater than or equal to 1. Alkyl groups may thus comprise 1 or more carbon atoms and generally, according to this invention comprise from 1 to 12, more preferably from 1 to 8 carbon atoms, and still more preferably from 1 to 6 carbon atoms. Alkyl groups within the meaning of the invention may be linear or branched. Examples of alkyl groups include but are not limited to methyl, ethyl, propyl, isopropyl, butyl, sec-butyl, isobutyl, tert-butyl, pentyl, sec-pentyl, isopentyl, hexyl and isohexyl.

The term “cycloalkyl” as used herein is a monovalent, saturated, or unsaturated monocyclic or bicyclic hydrocarbyl group. Cycloalkyl groups may comprise 3 or more carbon atoms in the ring and generally, according to this invention comprise from 3 to 10, more preferably from 3 to 8 carbon atoms, and still more preferably from 3 to 6 carbon atoms. Examples of cycloalkyl groups include but are not limited to cyclopropyl, cyclobutyl, cyclopentyl, and cyclohexyl.

The compounds for use of the invention containing a basic functional group may be in the form of pharmaceutically acceptable salts. Pharmaceutically acceptable salts of the compounds of the invention containing one or more basic functional groups include in particular the acid addition salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, cinnamate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Pharmaceutically acceptable salts of compounds of Formula I and subformulae may for example be prepared as follows:
(i) reacting the compound of Formula I or any of its subformulae with the desired acid; or
(ii) converting one salt of the compound of Formula I or any of its subformulae to another by reaction with an appropriate acid or by means of a suitable ion exchange column.

All these reactions are typically carried out in solution. The salt may precipitate from solution and be collected by filtration or may be recovered by evaporation of the solvent. The degree of ionization in the salt may vary from completely ionized to almost non-ionized.

The term “solvate” is used herein to describe a molecular complex comprising the compound of the invention and one or more pharmaceutically acceptable solvent molecules, for example, ethanol. The term “hydrate” is employed when said solvent is water.

The compounds of the invention include compounds of the invention as hereinbefore defined, including all polymorphs and crystal habits thereof, prodrugs and isomers thereof (including optical, geometric and tautomeric isomers) and isotopically-labeled compounds of the invention.

In addition, although generally, with respect to the salts of the compounds of the invention, pharmaceutically acceptable salts are preferred, it should be noted that the invention in its broadest sense also includes non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of the invention.

The term “patient” refers to a warm-blooded animal, more preferably a human, who/which is awaiting or receiving medical care or is or will be the object of a medical procedure.

The term “human” refers to subjects of both genders and at any stage of development (i.e. neonate, infant, juvenile, adolescent, adult). In one embodiment, the human is an adolescent or adult, preferably an adult.

The terms “treat”, “treating” and “treatment”, as used herein, are meant to include alleviating or abrogating a condition or disease and/or its attendant symptoms.

The term “therapeutically effective amount” (or more simply an “effective amount”) as used herein means the amount of active agent or active ingredient which is sufficient to achieve the desired therapeutic or prophylactic effect in the individual to which it is administered.

The term “administration”, or a variant thereof (e.g. “administering”), means providing the active agent or active ingredient, alone or as part of a pharmaceutically acceptable composition, to the patient in whom/which the condition, symptom, or disease is to be treated.

By “pharmaceutically acceptable” is meant that the ingredients of a pharmaceutical composition are compatible with each other and not deleterious to the patient thereof.

The term “excipient” as used herein means a substance formulated alongside the active agent or active ingredient in a pharmaceutical composition or medicament. Acceptable excipients for therapeutic use are well known in the pharmaceutical art, and are described, for example, in Remington's Pharmaceutical Sciences, 21^{st} Edition 2011. The choice of excipient can be selected with regard to the intended route of administration and standard pharmaceutical practice. The excipient must be acceptable in the sense of being not deleterious to the recipient thereof. The at least one pharmaceutically acceptable excipient may be for example, a binder, a diluent, a carrier, a lubricant, a disintegrator, a wetting agent, a dispersing agent, a suspending agent, and the like.

The term “pharmaceutical vehicle” as used herein means a carrier or inert medium used as solvent or diluent in which the pharmaceutically active agent is formulated and/or administered. Non-limiting examples of pharmaceutical vehicles include creams, gels, lotions, solutions, and liposomes.

The term “neurodegenerative disease” as used herein refers to the pathological condition in subjects that is characterized by the progressive loss of structure and/or function of neurons, which may ultimately involve cell death. Preferred neurodegenerative diseases in the context of the invention include motor neuron disease, in particular amyotrophic lateral sclerosis, and frontotemporal dementia.

The term “motor neuron disease” as used herein refers to a group of rare neurodegenerative disorders that selectively affect motor neurons, the cells which control voluntary muscles of the body. A preferred motor neuron disease in the context of the invention is amyotrophic lateral sclerosis.

The term “mitochondrial disorders” as used herein refers to pathologies associated with respiratory chain deficiency. Preferred mitochondrial disorders in the context of the invention include myopathy and cardiomyopathy.

The present invention will be better understood with reference to the following examples and figures. These examples are intended to be representative of specific embodiments of the invention, and are not intended as limiting the scope of the invention.

### FIGURES

FIG. 1: Negative controls for PLA analysis. Negative control experiments, using one antibody only, were performed in parallel to check the absence of PLA signal. P1, patient fibroblasts. IMMT, mitofilin.

FIG. 2: NFX toxicity and effect on cell growth. A. Toxicity of NFX. Control and patient fibroblasts (P1, P2) were treated during 72 h with DMSO or rising doses of NFX before cell counting. The percentage of living cells is expressed as percentage of DMSO control. B. Summary of NFX IC₅₀ on control and patient fibroblasts. C. Growth rate of control and patient (P1, P2) fibroblasts. Cell growth was measured during 72 h using the Xcelligence technology. D. Effect of NFX treatment on patient cell growth. 24 hours after seeding, cells were treated with DMSO, NFX 0.5 μM, 0.7 5 M or 1 μM. Cell growth was measured during 72 h using the Xcelligence technology.

FIG. 3: Treatment with NFX rescues the mitochondrial network fragmentation and restores the ultrastructure of mitochondrial cristae in patient fibroblasts A. Control or patient (P1, P2) fibroblasts were treated with DMSO, NFX 1 μM or 5 μM during 72 h before analysis of the mitochondrial network with by confocal microscopy. Mitochondria were stained with MitoTracker. P1 and P2 correspond to patient V-10 (Bannwarth et al., Brain, 2014, 137, 2329-2345) and IV-3 (Genin et al., EMBO Mol. Med., 2016, 8, 58-72), respectively. Both carry the p.Ser59leu variant (CHCHD10^{S59L/+}). B. Quantification of mitochondrial length in treated fibroblasts. Randomly selected individual 30 cells per condition were analyzed from 3 independent experiments. Values are mean±SEM. *: p<0.5; **: p<0,1; ***: p<0.01. C. Representative images of mitochondrial morphology by electron microscopy (EM), 72 h after treatment. D. Quantification of mitochondrial morphology observed in C. Mitochondria were classified in three categories <<normal>> (numerous and well organized cristae), <<intermediary>> (fewer cristae with mild disorganization) and <<abnormal>> (few or no cristae, disorganized). n=2 independent experiments (99 to 176 mitochondria per experiment). E. Duolink PLA between OPA1 and IMMT in Control and patient (P1, P2) fibroblasts treated with DMSO or NFX 5 μM, observed by confocal microscopy. Mitochondria were stained with MitoTracker. F. Duolink spots per cell were quantified for 20 randomly selected individual cells per each condition from 2 independent experiments. Values are mean±SEM. *: p<0.5; ***: p<0.001; n.s.: non significant.

FIG. 4. Treatment with NFX rescues the loss of mitochondrial cristae and decreases the caspase-dependent cell death in iPSC-derived patient motor neurons. A. Control (C1, C2) and patient (P1) motor neurons were treated with DMSO or NFX 0.5 μM during 72 h before analysis by electron microscopy. B. Quantification of mitochondrial morphology observed in A. n=2 independent experiments (15 to 137 mitochondria per experiment). Values are mean±SD. C. Control (C1 and C2) and patient (P1) motor neurons were treated with DMSO, glutamate 100 μM or glutamate 10 0 μM+NFX 0.5 μM prior to DEVDase activity measurement. Values are mean±SEM. *: p<0.5; **: p<0.1; ***: p<0.01.

FIG. 5. Treatment with NFX compensates the length of the mitochondrial network in patient fibroblasts with primary MICOS disassembly. A. BN-PAGE analysis showing MICOS state in control (C) and patient (P3, P4) fibroblasts. P3 and P4 patients carry loss of function mutations in the MIC13/QIL1 gene. Mitochondrial proteins from fibroblasts were analyzed by BN-PAGE with an anti mitofilin antibody. B. Control or patient (P3, P4) fibroblasts were treated with DMSO or NFX 5 μM during 72 h before analysis of the mitochondrial network with by confocal microscopy. Mitochondria were stained with MitoTracker. C. Quantification of mitochondrial length in treated fibroblasts. Randomly selected individual 30 cells per condition were analyzed from 3 independent experiments. Values are mean±SEM. **: p<0.1; ***: p<0.01; n.s: non significant.

FIG. 6. Effects of NFX analogs on yeast and human cellular models. Effects of Nitrofurantoin (NFI), a NFX structural analog, on patient fibroblasts. A. Toxicity of NFI. Control fibroblasts were treated during 72 h with DMSO or rising doses of NFI before cell counting. The percentage of living cells is expressed as percentage of DMSO control. B. Control or patient (P1, P2) fibroblasts were treated with DMSO or NFI 5 μM or 15 μM during 72 h before analysis of the mitochondrial network with by confocal microscopy. Mitochondria were stained with MitoTracker. C. Quantification of mitochondrial length in treated fibroblasts. Randomly selected individual 30 cells per condition were analyzed from 3 independent experiments. Values are mean±SEM. **: p<0.1; n.s: non significant.

FIG. 7: Effects of Nifurtoinol (NFO), a NFX structural analog, on patient fibroblasts. A. Toxicity of NFO. Control fibroblasts were treated during 72 h with DMSO or rising doses of NFO before cell counting. The percentage of living cells is expressed as percentage of DMSO control. B. Control or patient (P1, P2) fibroblasts were treated with DMSO or NFO 1 μM or 5 μM during 72 h before analysis of the mitochondrial network with by confocal microscopy. Mitochondria were stained with MitoTracker. C. Quantification of mitochondrial length in treated fibroblasts. Randomly selected individual 30 cells per condition were analyzed from 3 independent experiments. Values are mean±SEM. n.s: non significant.

FIG. 8: The effects of NFX on mitochondrial network in patient cells are not STAT3-dependent. A. Decrease of STAT3 expression level after NFX treatment. Control and patient (P1) fibroblasts were treated with DMSO, NFX 1 μM or 5 μM during 72 h before analysis of STAT3, pSTAT3Tyr705 and pSTAT3Ser727 protein levels by western blot. Antibodies against HSP60 and GAPDH were used for loading controls. B. Quantification of the blot presented in A. Values are mean±SEM. *: p<0.5; **: p<0.1; ***: p<0.01. C. Western blot on control and patient (P1, P2) fibroblast extracts in basal conditions, using antibodies against STAT3, pSTAT3Tyr705 and pSTAT3Ser727. Antibodies against HSP60 and GAPDH were used for loading controls. D. Quantification of the blot presented in C. Values are mean±SEM. *: p<0,5; **: p<0.1 E. Control or patient (P1, P2) fibroblasts were transfected with scrambled siRNA (siScr), as negative control, or STAT3 siRNA (siSTAT3) for experiments reported in FIG. 6F-G. Representative western blot of STAT3 expression level 72 h after transfection. Antibodies against HSP60 and GAPDH were used for loading control. F. Representative images of mitochondrial network from transfected fibroblasts. Mitochondria were stained with MitoTracker. G. Quantification of mitochondrial length in transfected fibroblasts. Randomly selected individual 30 cells per each transfection were analyzed from 2 independent experiments. Values are mean±SEM. *. p<0.5; **: p<0.1.

FIG. 9. Strategy used to identify NFX cellular targets. A. Affinity chromatography coupled with mass spectrometry to identify potential interactors of NFX. B. Patterns obtained after affinity chromatography done with NFX-beads compared to control magnetic beads. The * represent the NFX-beads specific bands or bands with a higher intensity.

FIG. 10. The effects of NFX on mitochondrial network in patient cells are KIF5B dependent. A. Control or patient (P1, P2) fibroblasts were transfected with scrambled siRNA (siScr), as negative control, or KIF5B siRNA (siKIF5B) and treated with DMSO or NFX 5 μM during 72h. Representative western blot of KIF5B expression level 72 h after transfection. Antibodies against HSP60 and GAPDH were used for loading controls. B. Quantification of mitochondrial network length in transfected fibroblasts. Randomly selected individual 30 cells per each transfection were analyzed from 2 independent experiments. Values are mean±SEM. *. p<0.05; **: p<0.01.; NS: non significant.

FIG. 11. NFX is able to improve the Mic60/Miro1 proximity in CHCHD10^{S59L/+} fibroblasts. A. Duolink PLA between Mic60 and Miro1 in control and patient (P1, P2) fibroblasts treated with DMSO or NFX 5 μM, observed by confocal microscopy. Mitochondria were stained with MitoTracker. B. Duolink spots per cell were quantified for 25-35 randomly selected individual cells per each condition for 2 different experiments. Values are mean±SEM. ***: p<0.001; ****: p<0.0001; ns: non significant.

### EXAMPLES

### Abbreviations

BDNF: Brain-Derived Neurotrophic Factor;
BSA: Bovine Serum Albumine;
CHIR99021: 6-[[2-[[4-(2,4-Dichlorophenyl)-5-(5-methyl-1H-imidazol-2-yl)-2-pyrimidinyl]amino]ethyl]amino]-3-pyridinecarbonitrile;
CNTF: Ciliary Neurotrophic Factor;
DAPT: (2S)—N-[(3,5-Difluorophenyl)acetyl]-L-alanyl-2-phenyl]glycine 1,1-dimethylethyl ester;
DMEM: Dulbecco's Modified Eagle Medium;
DMSO: Dimethylsulfoxide;
EB: Embyoid Body;
EM: Electron Microscopy;
FBS: Fetal Bovine Serum;
GDNF: Glial cell-derived Neurotrophic Factor;
IGF: Insulin-like Growth Factor 1;
iPSC: Induced Pluripotent Stem Cells;
LDN-193189:4-[6-[4-(1-Piperazinyl)phenyl]pyrazolo[1,5-a]pyrimidin-3-yl]quinoline dihydrochloride;
MICOS: Mitochondrial contact site and Cristae Organizing System;
NFL: Nitrofurantoin;
NFO: Nifurtoinol;
NFX: Nifuroxazide;
PBS: Phosphate-Buffered Saline;
PFA: Paraformaldehyde;
PLA: Proximity Ligation Assay;
PSF: Point Spread Function;
RPMI buffer: Roswell Park Memorial Institute buffer;
RT: Room Temperature;
SAG: Smoothened Agonist
SB431542: 4-[4-(1,3-benzodioxol-5-yl)-5-(2-pyridinyl)-1H-imidazol-2-yl]benzamide; SEM: Standard Error of the Mean;
Y-27632: trans-4-[(1R)-1-Aminoethyl]-N-4-pyridinylcyclohexanecarboxamide dihydrochloride;
YNB: Yeast Nitrogen Base;
YPGly: Yeast respiratory medium.

### Materials and Methods

### Cell Culture

Yeasts were grown in synthetic complete media (0.19% YNB without amino acids and 0.5% NH₄SO₄) supplemented with 1 g/L of dropout mix with amino acids or bases necessary to complement the auxotrophies. During the screening, the yeasts were grown on YPGly medium (1% yeast extract, 1% bactopeptone, 2% agar, 2% glycerol).

Skin punches were obtained from patients after informed consent. Primary fibroblast cultures were established using standard procedures in RPMI supplemented with 10% Fetal Bovine Serum (FBS), 45 μg/mL uridine and 275 μg/mL sodium pyruvate (Bannwarth et al., 2014). Primary fibroblast cultures were maintained in Dulbecco's Modified Eagle Medium supplemented with 10% FBS, penicillin (100 U/mL) and streptomycin (0.1 mg/mL) at 37° C. in a humidified incubator with 5% CO₂.

### Proliferation Measurement

The proliferation was measured using the Xcelligence machine (ACEA Bioscience). 50 μL of glucose medium (DMEM, 10% FBS, penicillin (100 U/mL) and streptomycin (0.1 mg/mL)) was added in the well of an Xcelligence 16 well plate to realise the blank of the machine. After trypsination, the cells were counted and resuspended in glucose medium at a concentration of 100 000 cells/mL and 50 μL of cells were plated per condition in the Xcelligence 16 well plates. The impedence as a proxy for cell division was followed during 72 h thanks to the RTCA software 2.0 and plotted thanks to the software GraphpadPrism 8 (GraphPad Software).

### Toxicity Assay

Cells were plated in six well plates in glucose medium (DMEM, 10% FBS, penicillin (100 U/mL) and streptomycin (0.1 mg/mL)) and incubated at 37° C. in a humidified incubator with 5% CO₂. After 24h, one well was treated with DMSO, the other wells were treated with rising concentrations of NFX, NFI or NFO. 72 h after the treatment, the cells were trypsinized, stained with Trypan blue and counted with a Malassez cell. The ratio of each concentration compared to the DMSO control has been plotted and the IC₅₀ has been calculated using the nonlinear regression on the software GraphpadPrism 8.

### Mitochondrial Network Analysis

Cells were incubated with MitoTracker red (Invitrogen) as previously described (Genin et al., EMBO Mol. Med., 2016, 8, 58-72). Fibroblasts were plated on a glass slide in a 60 mm dish. For the mitochondrial staining, the fibroblasts were incubated with MitoTracker Red (Invitrogen) (100 nM) during 15 minutes at 37° C. Then the medium was replaced by fresh medium for two hours at 37° C. The cells were fixed using Paraformaldehyde (Electron Microscopy Science) (4%) diluted in medium, 20 minutes at 37° C. The cells were then washed with PBS and mounted with Prolong Gold Antifade Reagent (Molecular Probes). The slides were analysed using a confocal microscope (Zeiss LSM 880). 30 cells were randomly selected and pictured. The images were deconvoluted with Huygens Essential Software™ (Scientific Volume Imaging) using a theoretically calculated point spread function (PSF) for each of the dyes. All selected images were iteratively deconvolved with maximum iterations scored 40 and a quality threshold at 0.05. The deconvolved images were used for quantitative mitochondrial network analysis with Huygens Essentiel Software™ with the following standardised set of parameters: threshold=15% and seed=10% for each cell types and garbage=10. The quantitative data were further analysed in Microsoft Excel and GraphPad Prism 8 (GraphPad Software). Mitochondrial network length was quantified for 30 randomly-selected individual cells. Data are represented as mean±S.E.M. Statistical analyses were performed by Student's unpaired t-test using GraphPad Prism 8 (GraphPad Software).

### Electron Microscopy

Cells were seeded on 24 wells/plate, fixed with 1.6% glutaraldehyde in 0.1 M cacodylate buffer (pH 7.4) for 2 hours, rinsed and postfixed in 1% osmium tetroxide and 1% potassium ferrocyanide in 0.1 M cacodylate buffer before to processed for ultrastructure as previously described (Bannwarth et al., Brain, 2014, 137, 2329-2345). The protocol for contrasting sections was slightly modified, using a 5% Lanthanide salt solution (a mixture of Gadolinium acetate and Samarium acetate) for 7 minutes instead of uranium acetate. The cells were examined under a JEOL 1400 transmission electron microscope. The ultrastructure of mitochondria was classified in three categories. Normal: the mitochondria have numerous, parallel and well organized cristae. Intermediary: the mitochondria have less cristae, they show few round or swollen cristae or have a mild disorganisation. Abnormal: the mitochondria have swollen, disorganized or no cristae. More than 100 mitochondria were classified per condition.

### In Situ Proximity Ligation Assay (PLA)

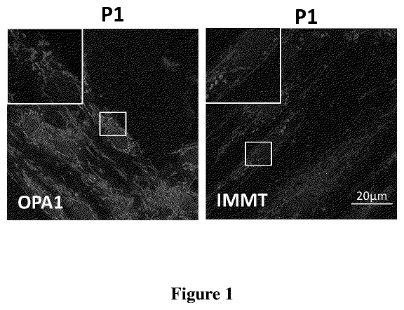

Fibroblasts were seeded in 16-well Lab-Tek chambers slides (Nunc). For mitochondrial staining, cells were incubated in a 100 nM solution of MitoTracker Red CMXRos (Invitrogen) for 15 min. Cells were fixed with PFA 4% for 20 min at 37° C. and permeabilised with 2% Triton X-100 for 10 min. Then coverslips were saturated with 5% BSA for 45 min at RT. The following antibodies were used in PLA assay: rabbit anti OPA1 and mouse anti IMMT (listed in Table 2 below). All antibodies were diluted with PBS-BSA 5%. Fibroblasts were incubated in the presence of convenient couple of primary antibodies for 1 h at RT. After PBS washes, incubation with appropriate PLA probe (PLA probe anti rabbit MINUS, PLA probe anti mouse PLUS), hybridization, ligation and amplification were done using the DuoLink In Situ Detection Reagents Green (Olink Biosciences) following manufacturer's instructions. Finally, the samples were mounted on glass slides using Prolong Gold Antifade Reagent (Molecular Probes). Images were captured with a ZEISS LSM 880 confocal laser-scanning microscope and analyzed using Huygens Essential Software™ (Scientific Volume Imaging). Data are represented as mean±SEM. Negative control experiments (with one antibody omitted) were performed in parallel and checked to result in the absence of PLA signal (FIG. 1).

TABLE 2

| Antibody | Reference | Company | Species | Dilution |
|---|---|---|---|---|
| GAPDH | 10494-1-AP | Proteintech | Rabbit | 1/50 000 |
| HSP60 | 15828-1-AP | Proteintech | Rabbit | 1/50 000 |
| IMMT | sc-390736 | Santa-Cruz | Mouse | 1/1000 |
| | | Biotechnology | | |
| STAT3 | sc-8019 | Santa-Cruz | Mouse | 1/500 |
| | | Biotechnology | | |
| pSTAT3Tyr705 | #9131 | Cell Signaling | Rabbit | 1/500 |
| pSTAT3Ser727 | #9134 | Cell Signaling | Rabbit | 1/500 |

iPSC Generation and Motor Neuron Differentiation

iPSC clones were generated from human fibroblasts as previously described (Genin et al., Acta Neuropathol., 2019, 138, 123-145). Motor neurons were differentiated from iPSC using previously described and validated protocols with some modifications. Briefly, iPSC clones were detached by Accutase treatment to form small clusters. Cells were transferred in Neuronal basic medium (DMEM/F12 plus Neurobasal medium with N2 and B27 supplement without vitamin A) supplemented with 40 μM SB431542 (Tocris Bioscience), 0.2 μM LDN-193189 (Stemgent), 3 μM CHIR99021 (Tocris Bioscience), and 5 μM Y-27632 (Merck Millipore) to induce EB formation. From day 3 on, 0.1 μM retinoic acid (Sigma) and 500 nM SAG (Merck Millipore) were added to the neuronal basic medium and medium was changed every 2 days. From day 9 on, BDNF (10 ng/mL, Miltenyi Biotec) and GDNF (10 ng/ml, Miltenyi Biotec) and DAPT (20 μM, Tocris Bioscience) were added. On day 10, floating clusters were dissociated into single cells using 0.05% trypsin (Gibco™). Motor neurons were seeded on laminin (20 μg/mL)-coated 24-well plates at the density of 0.2-2×105 cells per well. From day 17 on, the cells were switched to motor neuron maturation medium supplemented with BDNF, GDNF, CNTF and IGF (each 10 ng/mL, Miltenyi Biotec) to keep long-term cultures. Media were changed every 2 days by replacing half of the medium. For caspase activity measurement, motor neurons were treated either with 0.5 M Staurosporine (Sigma) for 24 h or 50-100 μM glutamate (Sigma) for 48h.

### Caspase Activity Measurement

Cells were treated either with 0.5 μM Staurosporine (Sigma) for 24 h or 50-100 μM glutamate (Sigma) for 48 h and caspase activation was analyzed by DEVDase activity measurement as previously described (Villa et al., 2017). Briefly, cells were lysed and after normalized to protein content, lysates were loaded into a black 96-well plate in the presence of 0.2 mmol/l of the caspase-3 substrate Ac-DEVD-AMC. Caspase activity was measured using a fluoroscan at 460 nm, and specific activity was expressed as the change in absorbance per minute per milligram protein.

### BN-PAGE

BN-PAGE on human fibroblasts were performed using NativePAGE reagent (Invitrogen) according to the standard procedure. Briefly, cells were lysed 15 min on ice in NativePAGE sample buffer 1× containing 1% digitonin or 2% digitonin for the extraction of MICOS complex, respectively. The lysate was clarified by centrifugation at 20,000×g for 30 min at 4° C. Fifteen micrograms of proteins were separated by Blue Native-PAGE Novex 4-16% Bis-Tris gel (Thermo Fisher Scientific). Gels were stained with Coomassie Brilliant Blue. Proteins from the resulting gels were transferred to PVDF membranes (Millipore) and analyzed by western blotting with relevant antibodies.

siRNA Transfection

siRNA transfections were performed using Lipofectamine RNAiMAX reagent (Invitrogen) following manufacturer's instructions. siRNAs (listed in Table 3 below) were purchased from Dharmacon. Primary fibroblasts were transfected with 55 pmol of siRNA to a final concentration of 55 nM and analysed 72 h post-transfection.

TABLE 3

| siRNA | Reference | Company |
|---|---|---|
| siGENOME Human | M-031920-00-0005 | Dharmacon |
| C19orf70 siRNA | | |
| siGENOME Non- | D-001206-14-05 | Dharmacon |
| Targeting siRNA | | |

### Western Blot Analysis

The concentration of proteins was determined using the Pierce BCA assay kit (Thermo Fisher Scientific). 20 μg of total protein extracts were separated on acrylamide-SDS gels and transferred to PVDF membranes (Millipore). Specific proteins were detected by using different antibodies listed in Table 2 above. Signals were detected using a chemoluminescence system (Immobilon Western HRP Chemilumiscent substrates, Millipore). ImageJ was used to quantify protein signals.

### Nifuroxazide Treatment

Nifuroxazide (46494-100MG Sigma) was resuspended in DMSO at 50 mM and stocked at −20° C. For each treatment, the Nifuroxazide was diluted in complete medium (Dulbecco's Modified Eagle Medium supplemented with 10% FBS, penicillin (100 U/mL) and streptomycin (0.1 mg/mL)) at the desired concentration. The cell medium was discarded and replaced by the medium with Nifuroxazide. 72 h hours after treatment, the cells were analysed.

### Statistical Analysis

Statistical analysis was done with Student's t-test and Mann-Whitney's test. The quantitative data were analysed using Microsoft Excel and Graphpad Prism 8 (Graphpad Software). Data are expressed are mean±SEM. p-values: *<0.05, **<0.01, ***<0.001, n.s.=non significant.

### Generation of NFX Beads

Magnetic beads to which NFX has been covalently coupled were synthetized according the reaction scheme 1 below:

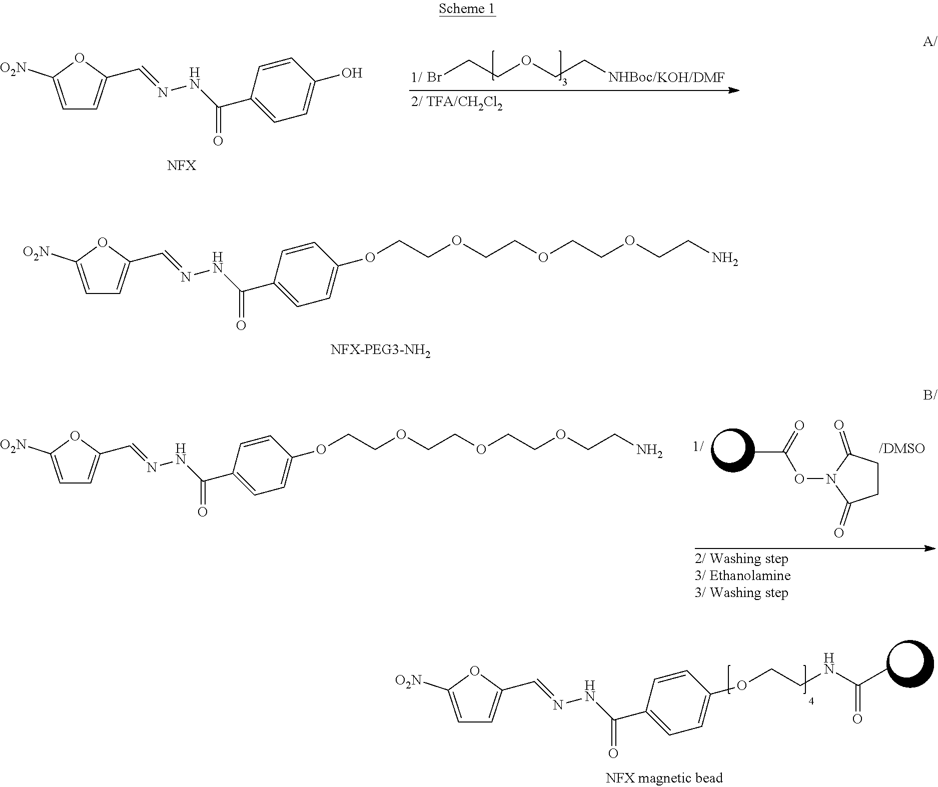

### Synthesis of NFX-PEG3-NH₂

### tert-butyl(E)-(2-(2-(2-(2-(4-(2-((5-nitrofuran-2-yl)methylene)hydrazine-1-carbonyl)phenoxy)ethoxy)ethoxy)ethoxy)carbamate: NFX-PEG3-NHBoc

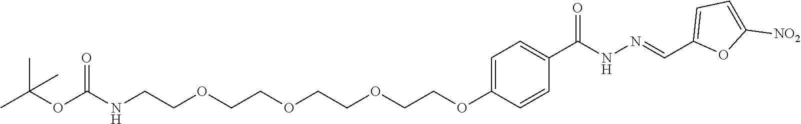 [SMILES: CC(C)(C)OC(=O)NCCOCCOCCOCCOc1ccc(C(=O)N/N=C/c2ccc([N+](=O)[O-])o2)cc1]

To a solution of nifuroxazide (77 mg, 0.28 mmol, 2 eq.) in dry DMF (3 mL) was added KOH (31 mg, 0.56 mmol, 4 eq.) and the mixture was stirred at room temperature. After 1 h was added NHBoc-PEG-Br (50 mg, 0.14 mmol, 1 eq.) and the reaction was stirred at room temperature overnight. The solvent was removed, water was added and extracted with DCM. The organic phase was dried over MgSO₄ and the solvent removed under reduced pressure. The crude residue was purified by flash chromatography to afford the compound as a yellow solid (37 mg, 48%).

### 4-(2-(2-(2-(2-aminoethoxy)ethoxy)ethoxy)ethoxy)-N′-((5-nitrofuran-2-yl)methylene)benzohydrazine: NFX-PEG3-NH₂

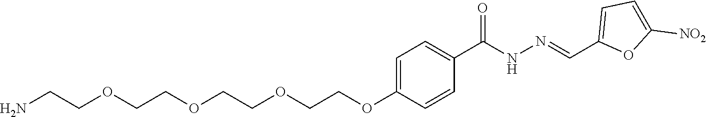 [SMILES: NCCOCCOCCOCCOc1ccc(C(=O)N/N=C/c2ccc([N+](=O)[O-])o2)cc1]

A solution of NFX-PEG-NHBoc (20 mg, 0.036 mmol, 1 eq.) in dichloromethane (500 μL) was treated with TFA (35 μL, 0.36 mmol, 10 eq.) for 5 h at room temperature. The solvents was removed in vacuo and the crude was co-evaporated with toluene. The crude residue was purified by flash chromatography to afford the compound as a yellow solid (15 mg, 94%).

### Coupling Procedure

This protocol was established for coupling 0.16 mmol of NFX-PEG3-NH₂ to 1 mL NHS Pierce Activated Magnetic Beads in a 1.5 mL microtube. The reaction can be scaled up or down linearly if required.

1 mL of non-coupled NHS Pierce Activated Magnetic Beads was dispensed into a 1.5 mL microtube and the tube was placed in a magnetic stand. The supernatant was removed and the beads were washed two times with 1 mL of dry DMSO each. NFX-PEG3-NH₂ (74 mg, 0.16 mmol, 1 eq.) was dissolved into 1 mL dry DMSO, the resulting solution was added to the beads with Et₃N (27 μL, 0.20 mmol, 1.2 eq.) and the resulting mixture was mixed by vortexing. The reaction mixture was incubated overnight at r.t. on a tube revolver rotator. The microtube was placed into a magnetic stand and the supernatant was discarded. The magnetic beads were washed two times with 1 mL of dry DMSO. Ethanolamine (49 μL, 0.82 mmol, 5 eq.) was added to the beads and mix by vortexing. The reaction mixture was incubated for 4 h at r.t. on a tube revolver rotator. The microtube was placed into a magnetic stand and the supernatant was discarded. The magnetic beads were washed two times with 1 mL of dry DMSO. The microtube was placed into a magnetic stand and the supernatant was discarded. The magnetic beads were washed two times with 1 mL of dry DMSO. The beads were then resuspended in 1 mL of N,N-dimethylacetamide. The resulting was kept at 4° C. until use.

### Affinity Chromatography

Fibroblasts were trypsinized and washed with PBS 1× then incubated in lysis buffer on ice during 20 minutes (Lysis buffer: Tris-HCl pH 7.5 50 mM, EDTA 2 mM, Triton X-100 0.1% supplemented with anti-protease and anti-phosphatase (Complete-Mini™—Roche). After centrifugation 15 minutes, 13 000 rpm at 4° C., supernatant was conserved and the protein concentration was determined using the Pierce BCA assay kit (Thermo Fisher Scientific).

10 μL of magnetic beads and NFX-beads were washed three times in Bead buffer before to be resuspended 100 μL of Bead buffer (Bead buffer: Tris-HCl pH 7.5 50 mM, NaCl 150 mM, Triton X-100 0.1%, supplemented with anti-protease and anti-phosphatase (Complete-Mini™—Roche)) thanks to a magnetic rack (DynaMag-2 Ref 12321D Life Technologies). 800 μg of proteins were mixed with 100 μL of diluted beads and the final volume was set to 800 μL with Bead buffer. The proteins and the beads were incubated 30 minutes with rotation in a cold room. Using a magnetic rack, the beads were separated from the protein extract and washed three time with Bead buffer before to be resuspended in 75 μL of Laemmli2X. Laemmli with beads was put 5 minutes at 95° C. to eluate the proteins and the beads were discarded using a magnetic rack. 35 μL of the eluted proteins were subjected to SDS-PAGE and silver coloration (ProteoSilver™ Silver Stain Kit—Sigma Aldrich). Bands of interest were extracted from the gel and subjected to mass spectrometry.

### Mass Spectrometry Analysis

Mass spectrometric identification and quantification of the pulled down proteins were performed as previously described (Shrivastava et al., EMBO, 2019, 38:e99871). The raw data were cleaned using the following pathway. First, only the proteins found to interact with the NFX coupled beads in the three replicates were kept for further analysis. Second, only the proteins with a total number of spectral counts superior to 20 were kept for further analysis. Third, only the proteins with a number of spectral counts in the NFX coupled beads five time superior to the beads alone were kept for the final result. The final proteins were then classified based on the fold chance and the p-value.

siRNA Transfection, Mitochondrial Network Analysis, Proximity Ligation Assay, NFX Treatment

All experiments were performed as described in the original patent by using KIF5B siRNA (ON-TARGET plus Human KIF5B (3799) siRNA—SMART pool, Ref L-008867-00-0005, Dharmacon—Horizon) and, antibodies against Miro1 (Rabbit, Invitrogen: PA5-72835) and Mic60 (Mouse, Abcam ref: ab110329).

### Results

### Nifuroxazide Rescues Mitochondrial Network Fragmentation and Cristae Abnormalities in CHCHD10^{S59L/+} Fibroblasts

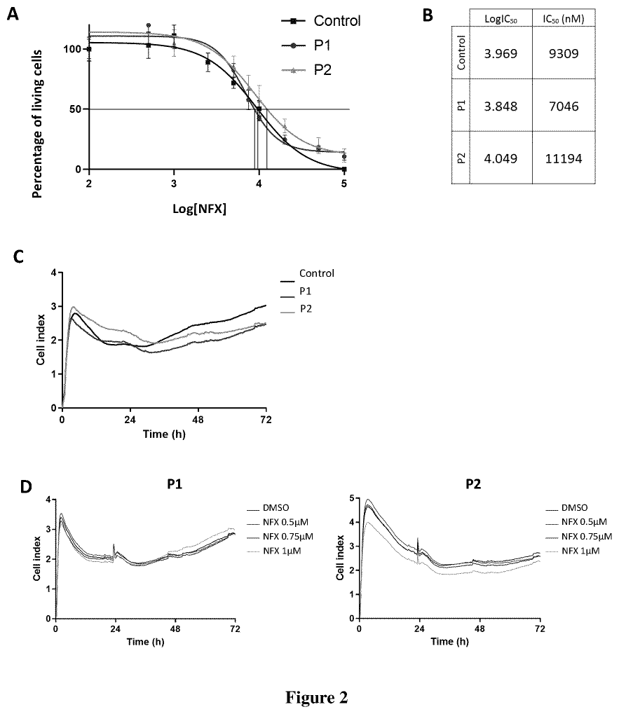

The effects of NFX on cultured skin fibroblasts from 2 patients carrying the p.Ser59Leu heterozygous variant in CHCHD10 was first studied. The toxicity of NFX was determined by following the proliferation of control and patient fibroblasts incubated with increasing drug concentrations. The calculated IC50 value was around 10 μM (FIG. 2A-B). Patient fibroblasts do not show a significant growth deficit compared to control cells (FIG. 2C) and increasing doses of NFX did not show any effect on the growth rate of these cells (FIG. 2D).

It has been previously showed that CHCHD10^{S59L/+} fibroblasts display both a fragmented mitochondrial network and abnormal mitochondrial morphology with loss of cristae (Bannwarth et al., Brain, 2014, 137, 2329-2345).

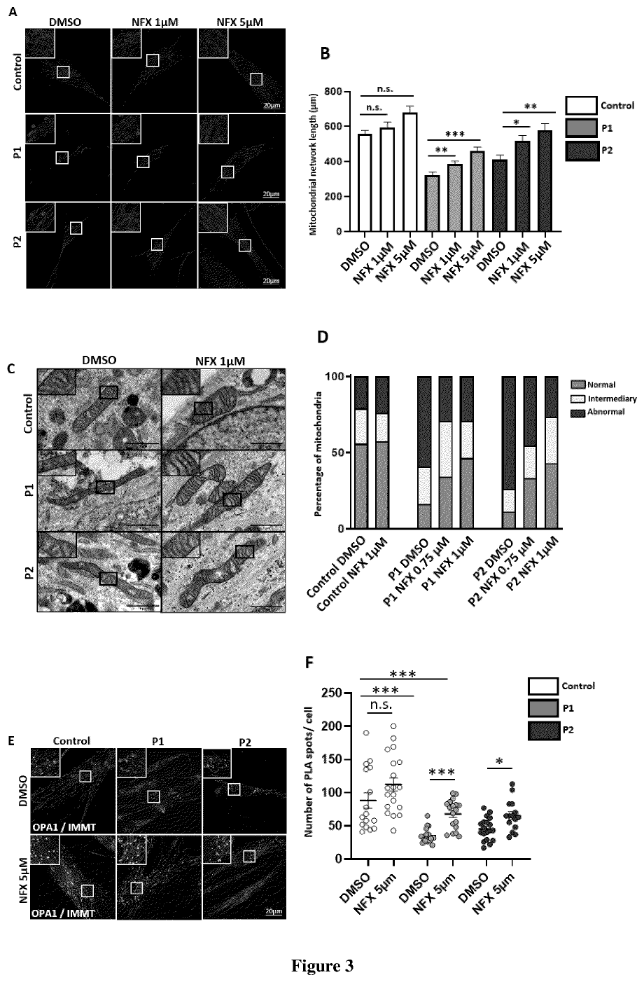

FIG. 3A-B shows that NFX treatment induces a significant elongation of the mitochondrial network in a dose-dependent manner. Moreover, EM analysis revealed that mitochondrial morphology and cristae shape were improved in patient fibroblasts after NFX treatment (FIG. 3C-D). CHCHD10 physically interacts with Mitofilin/Mic60, a key component of the MICOS complex that is destabilized in CHCHD10^{S59L/+} fibroblasts (Genin et al., EMBO Mol. Med., 2016, 8, 58-72; Liu et al., FASEB J., 2020, 34, 8493-8509). Mitofilin also interacts with OPA1, and together, they control cristae junction number and stability (Barrera et al., FEBS Letters, 2016, 590, 3309-3322; Glytsou et al., Cell Reports, 2016, 17, 3024-3034). Recently, we showed by co-IP analysis that the level of interaction between Mitofilin and OPA1 was decreased in CHCHD10^{S59L/+} fibroblasts compared to control cells. This result was confirmed by Proximity Ligation Assay (PLA) analysis showing a decreased proximity between these 2 proteins in patient cells compared to control fibroblasts (Genin et al, submitted). Treatment with NFX led to a significant increase of the number of PLA spots in patient fibroblasts suggesting that the molecule is able to rescue the interaction between OPA1 and Mitofilin thus contributing to restore the ultrastructure of mitochondrial cristae (FIG. 3E-F). All of these results suggest that NFX rescues phenotypes associated with MICOS disassembly via a pathway that is conserved between yeast and humans.

Nifuroxazide has Positive Effects on Patient iPSC-Derived Motor Neurons

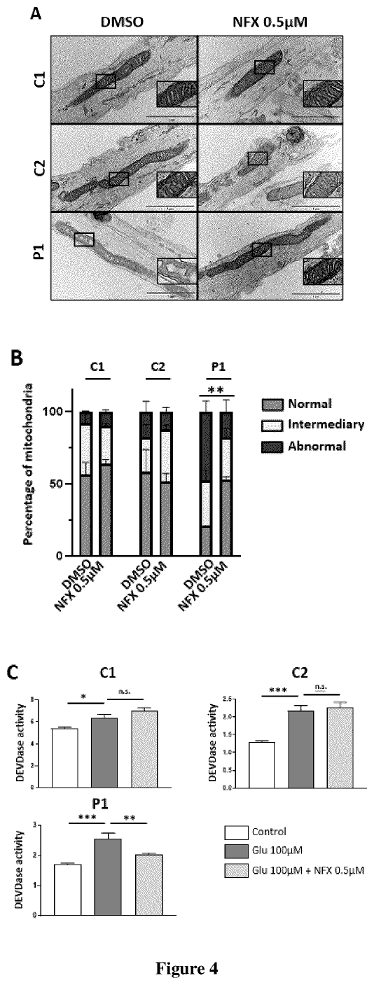

The effects of NFX on cellular models more relevant than fibroblasts for MND were then analyzed. Human iPSC cells from patients carrying the p.Ser59Leu mutation were generated and showed that they are able to differentiate into motor neurons. However, the CHCHD10^{S59L/+} iPSC-derived motor neurons display mitochondria with altered morphology. Furthermore, they are much more sensitive to caspase-dependent cell death than control neurons (Genin et al., Acta Neuropathol., 2019, 138, 123-145). By EM analysis, it was observed that mitochondrial morphology and cristae shape were improved after NFX treatment in patient motor neurons (FIG. 4A-B). Importantly, CHCHD10^{S59L/+} motor neurons were less sensitive to glutamate-induced caspase activation after NFX treatment while the molecule had no effect on control motor neurons (FIG. 4C).

Nifuroxazide Also Compensates the Length of the Mitochondrial Network in Patient Cells with Primary MICOS Disassembly

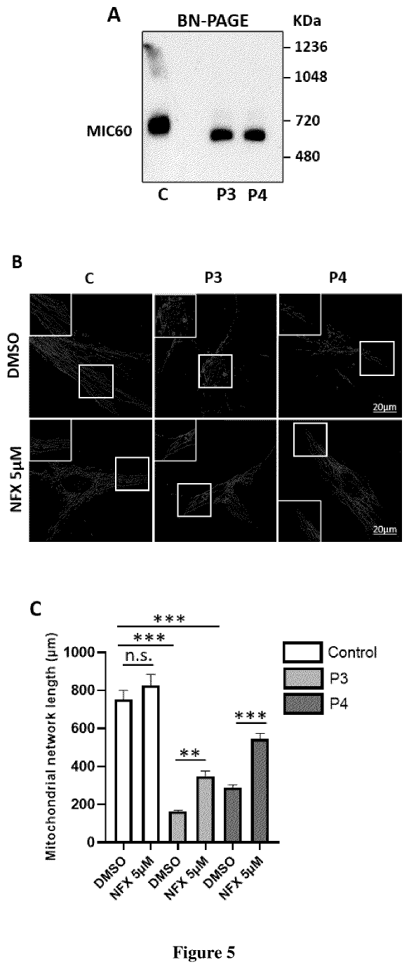

MICOS is composed of conserved subunits and MIC13/QIL1 is the structural ortholog of yeast Mix12 required for the stability of the complex (Guarani et al., Elife, 2015, 4, e06265; Huynen et al., Biochim. Biophys. Acta, 2016, 1863, 91-101). Variants responsible for MIC13/QIL1 loss of function are associated with severe mitochondrial encephalo-hepatopathy (Guarani et al., Elife, 2015, 4, e06265). Patient fibroblasts display MICOS complex disassembly and abnormal mitochondrial cristae (Guarani et al., Elife, 2015, 4, e06265; Kishita et al., Mol. Genet. Genomic Med., 2020, 8, e1427; Zeharia et al., Eur. J. Hum. Gen., 2016, 24, 1778-1782). The effects of NFX on fibroblasts of patients carrying loss of function mutations in MIC13/QIL1 were studied. Patient 3 (P3) carried a homozygous splice site mutation (c.30-1G>A) (Guarani et al., Elife, 2016, 5, e17163) and in patient 4 (P4), the disease was associated with a homozygous frameshift variant (c.143DupT; p.Ala51Argfs*32). BN-PAGE analysis performed on P3 and P4 fibroblasts confirmed the defect of MICOS stability (FIG. 5A). Confocal analysis after MitoTracker staining showed that both P3 and P4 cells display a fragmented mitochondrial network. Treatment with NFX led to a significant increase of the length of the mitochondrial network in these cells (FIG. 5B-C).

### Effects of Analogs of Nifuroxazide on Yeast and Human Cell Models

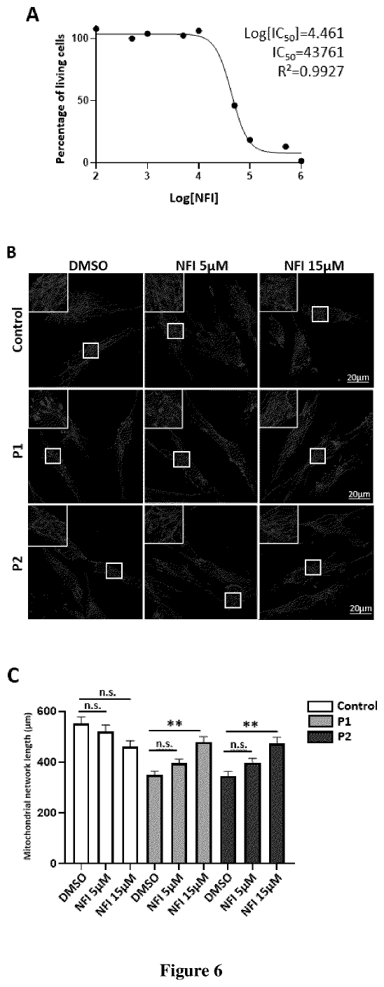

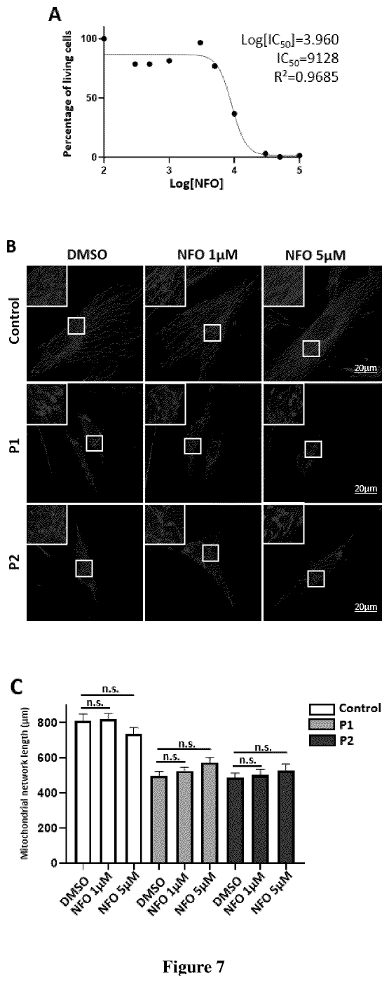

CHCHD10^{S59L/+} fibroblasts were treated with Nifurtoinol (NFO) and Nitrofurantoin (NFI) and their effect on mitochondrial dynamics of patient cells was analysed. It was observed that NFI, which showed a lower toxicity than that of NFX, was able to significantly increase the length of the mitochondrial network in CHCHD10^{S59L/+} fibroblasts (FIG. 6A-C). However, to achieve an equivalent mitochondrial network length, the doses of NFI needed were greater than those of NFX. FIG. 7A-C shows the results obtained for NFO.

### The Positive Effects of Nifuroxazide on Mitochondrial Settings in CHCHD10^{S59L/+} Cells are not STAT3-Dependent.

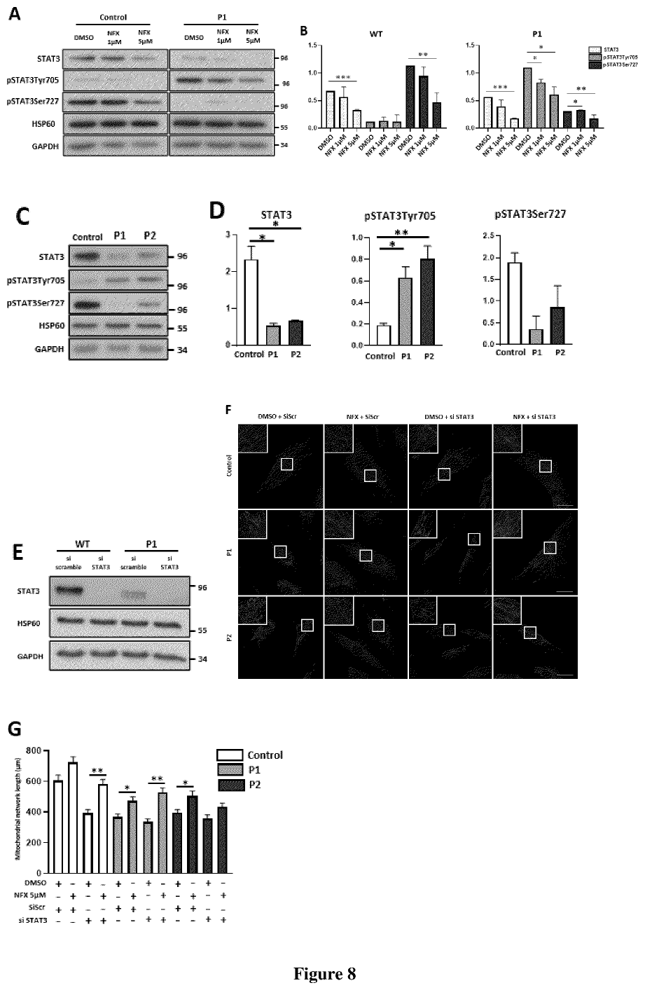

NFX is a potent inhibitor of STAT3 tyrosine phosphorylation. STAT3 is a transcription factor, activated by phosphorylation on Y705, which determines the ability to concentrate in the nucleus, and to bind DNA (Avalle et al., Int. J. Mol. Sci., 2018, 19, 2820; Nelson et al., Blood, 2008, 112, 5095-5102). Deregulation of STAT3 activity has been detected in tumors of different types contributing to tumor transformation, growth and progression. To verify that the improvement of mitochondrial parameters observed in patient cells was associated to a biological effect of NFX, STAT3 expression after NFX treatment was analysed. Both in patient and control fibroblasts, it was observed a dose-dependent decrease of STAT3 and pSTAT3Y705 after NFX treatment (FIG. 8A-B). Interestingly, it was observed that pSTAT3Y705 levels were significantly higher in patient fibroblasts than in control cells in basal conditions (FIG. 8 C-D). STAT3 also has non-canonical functions in the mitochondria, contributing to energy homeostasis under stress conditions, through its phosphorylation on a carboxy-terminal serine residue, serine 727. In order to determine whether the effects observed in patient fibroblasts after NFX treatment could be dependent on STAT3, STAT3 expression in CHCHD10^{S59L/+} fibroblasts was blunted. Transfection with STAT3 siRNA led to STAT3 depletion in control and patient cells without preventing the positive effect of NFX on mitochondrial network length in fibroblasts (FIG. 8E-G). These results suggest that the effect of NFX on mitochondrial dynamics in patient cells is not STAT3-dependent.

### KIF5B is a Cellular Target of Nifuroxazide

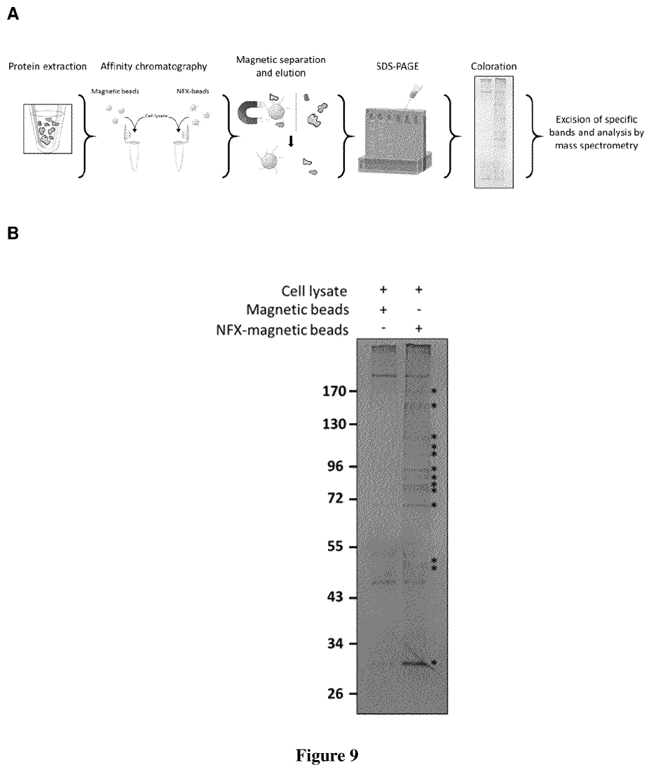

Identification of the NFX intracellular targets is a crucial step in understanding its mechanisms of action and to be able to improve its efficiency on a rational basis. To purify NFX-binding proteins, affinity chromatography purification was performed using magnetic beads. Protein extracts from human fibroblasts were incubated with magnetic beads alone or to which NFX has been covalently coupled (Scheme 1). Eluted fractions were subjected to SDS-PAGE electrophoresis and silver staining (FIG. 9A). The pattern was clearly different between eluted proteins from NFX-beads compared to those eluted form beads alone with 13 bands either appeared or were of a higher intensity with the NFX-beads (FIG. 9B). Excised bands were subjected to proteolytic digestion and mass spectrometry.

Among the 10 top NFX targets identified (Table 4), the inventors' attention was focused on KIF5B that plays a key role in transporting mitochondria in the anterograde direction along microtubules.

TABLE 4

| | | Accession | Mol. weight | Fold | |
|---|---|---|---|---|---|
| Protein | Gene | Number | [kDa] | change | p -value |
| Calcium/calmodulin-dependent | CAMK2D | Q13557 | 56 kDa | 803.33 | 5.83E−07 |
| protein kinase type II subunit delta | | | | | |
| ARF GTPase-activating protein GIT2 | GIT2 | Q14161 | 85 kDa | 433.33 | 2.11E−05 |
| E3 ubiquitin-protein ligase DTX3L | DTX3L | Q8TDB6 | 84 kDa | 386.67 | 1.26E−04 |
| Kinesin-1 heavy chain | KIF5B | P33176 | 110 kDa | 121.67 | 7.35E−06 |
| Pleckstrin homology-like | PHLDB2 | Q86SQ0 | 142 kDa | 91.67 | 1.90E−05 |
| domain family B member 2 | | | | | |
| Vigilin | HDLBP | Q00341 | 141 kDa | 77.14 | 5.88E−06 |
| Myotubularin-related protein 5 | SBF1 | O95248 | 208 kDa | 41.35 | 2.45E−05 |
| RNA-splicing ligase RtcB homolog | RTCB | Q9Y3I0 | 55 kDa | 40.98 | 1.15E−05 |
| Unconventional myosin-IXb | MYO9B | Q13459 | 243 kDa | 34.52 | 2.47E−04 |
| Mitogen-activated protein kinase | MAP4K4 | O95819 | 142 kDa | 29.76 | 3.04E−05 |

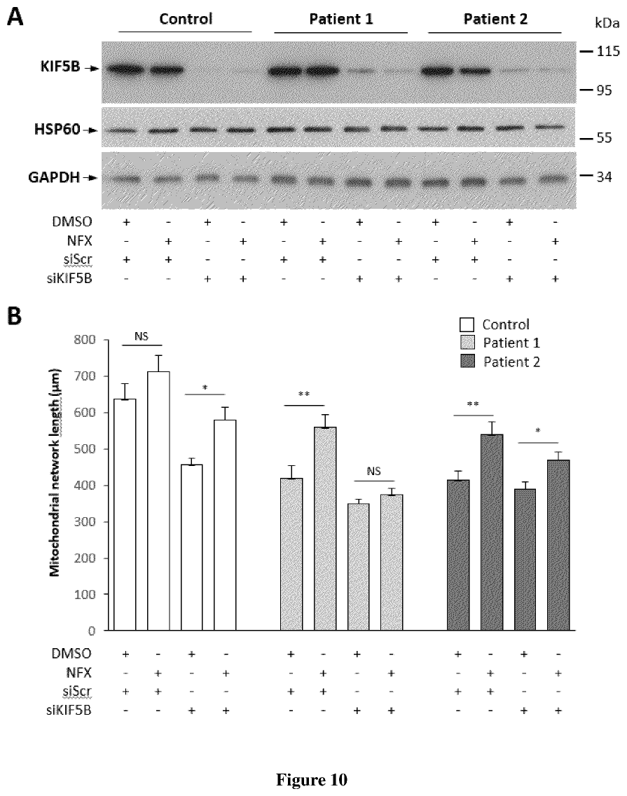

KIF5B binds to mitochondria via a complex composed of Miro1 and the TRAK adaptors. Miro, a GTPase anchored to the mitochondrial membrane, binds to TRAK proteins which in turns bind to KIF5B linking mitochondria to microtubules (Zhao et al., eLIFE, 2020, 9:e53456). Recently, it has been shown that Mic60/Mitofilin interacts with Miro1 and that MICOS, Miro1 and KIF5B are involved in both cristae organization and in the coordination of the transportation and of the proper distribution of nucleoids in the cell (Modi et al., Nat Comm, 2019, 10:4399; Qin et al., Nat Comm, 2020, 11:4471). In order to confirm that KIF5B is a cellular target of NFX, the inventors decided to blunt KIF5B expression in CHCHD10^{S59L/+} fibroblasts. Transfection with KIF5B siRNA led to KIF5B depletion in control and patient cells (FIG. 10A). It has been observed that NFX no longer has an effect on the mitochondrial network in patient cells after inactivation of KIF5B (FIG. 10B). These data suggest that the effect of NFX on mitochondrial dynamics in patient cells could be dependent on KIF5B that is a cellular target of the molecule.

### Nifuroxazide Increases the Interaction Between Mic60 and Miro1 in CHCHD10^{S59L/+} Cells

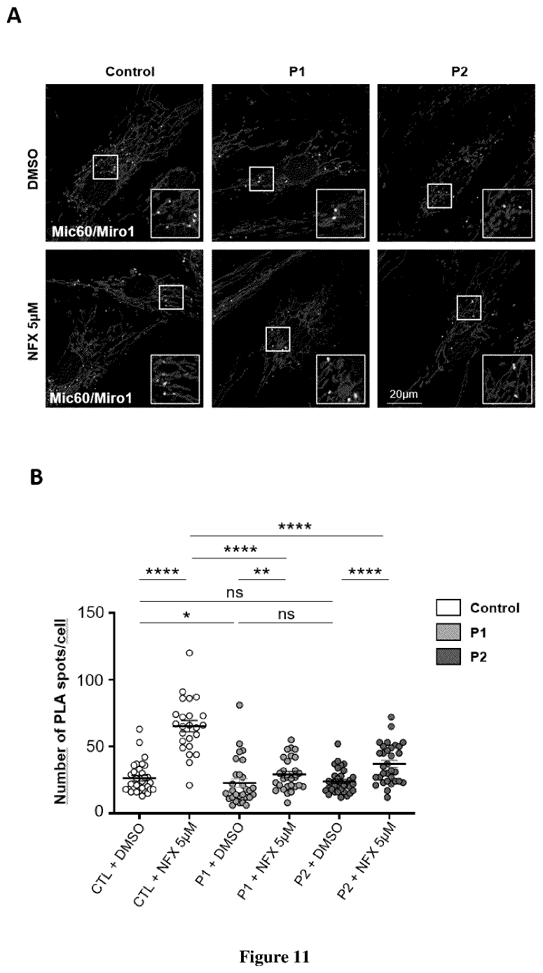

It has been shown that both cristae organization and mitochondria transport along microtubules require a direct interaction between Mic60, Miro1 and KIF5B (Modi et al., Nat Comm, 2019, 10:4399; Qin et al., Nat Comm, 2020, 11:4471). The inventors investigated whether the interaction between Mic60 and Miro1 could be altered in patient fibroblasts and whether NFX treatment could improve it, thus contributing in part to the improvement of cristae morphology that we observed. Therefore, PLA was performed to analyse the proximity between Mic60 and Miro1 in patient fibroblasts compared to control cells. A significant decrease of PLA spots was observed in patient cells, which suggests that the interaction between Mic60 and Miro1 is disturbed in CHCHD10^{S59L/+} cells (FIG. 11). Treatment with NFX led to a significant increase of the number of PLA spots in patient fibroblasts suggesting that the molecule is able to rescue the interaction between Mic60 and Miro1, thus contributing to restore the ultrastructure of mitochondrial cristae (FIG. 11).

1. A method of treating diseases associated with mitochondrial dysfunction, comprising the step of administering to a patient in need thereof a therapeutically effective amount of a compound of Formula I: 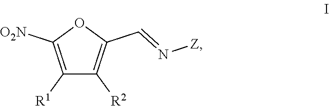
a pharmaceutically acceptable salt or a solvate thereof,
wherein
R¹ and R² are independently selected from H, C1-C6-alkyl, C3-C6-cycloalkyl and halogens, and
Z is selected from 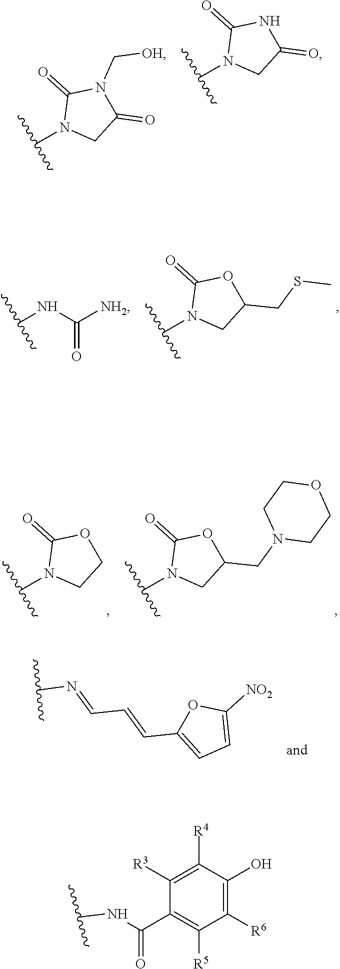
wherein R³, R⁴, R⁵ and R⁶ are independently selected from H, C1-C6-alkyl, C3-C6-cycloalkyl, OH and halogens.

2. The method according to claim 1, wherein R¹ and R² are H.

3. The method according to claim 1, wherein R³, R⁴, R⁵ and R⁶ are H.

4. The method according to claim 1, wherein the compound is selected from the group consisting of:
(E)-4-hydroxy-N′-((5-nitrofuran-2-yl)methylene)benzohydrazide);
(E)-3-(hydroxymethyl)-1-(((5-nitrofuran-2-yl)methylene)amino)imidazolidine-2,4-dione;
(E)-1-(((5-nitrofuran-2-yl)methylene)amino)imidazolidine-2,4-dione;
(E)-2-((5-nitrofuran-2-yl)methylene)hydrazine-1-carboxamide;
(E)-5-((methylthio)methyl)-3-(((5-nitrofuran-2-yl)methylene)amino)oxazolidin-2-one;
(E)-3-(((5-nitrofuran-2-yl)methylene)amino)oxazolidin-2-one;
(E)-5-(morpholinomethyl)-3-(((5-nitrofuran-2-yl)methylene)amino)oxazolidin-2-one; and
(1E,2E)-1-((E)-3-(5-nitrofuran-2-yl)allylidene)-2-((5-nitrofuran-2-yl)methylene)hydrazine.

5. The method according to claim 1, wherein the diseases associated with mitochondrial dysfunction are selected from mitochondrial disorders and neurodegenerative diseases.

6. The method according to claim 5, wherein the mitochondrial disorders are selected from myopathy and cardiomyopathy.

7. The method according to claim 5, wherein the neurodegenerative diseases are selected from motor neuron disease and frontotemporal dementia.

8. The method according to claim 7, wherein the motor neuron disease is amyotrophic lateral sclerosis.

9. A method of treating diseases associated with mitochondrial dysfunction, comprising the step of administering to a patient in need thereof a therapeutically effective amount of a pharmaceutical composition comprising a compound Formula I: 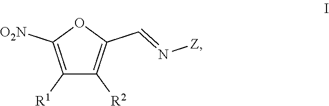
or a pharmaceutically acceptable salt or solvate thereof and at least one pharmaceutically acceptable excipient,
wherein
R¹ and R² are independently selected from H, C1-C6-alkyl, C3-C6-cycloalkyl and halogens, and
Z is selected from 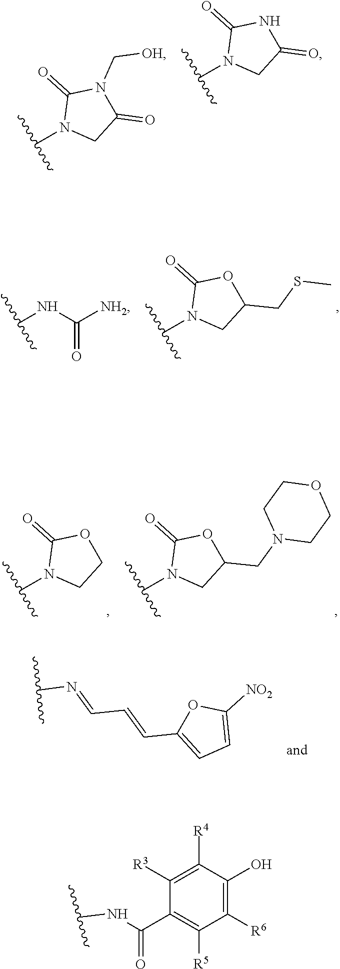
wherein R³, R⁴, R⁵ and R⁶ are independently selected from H C1-C6-alkyl, C3-C6-cycloalkyl, OH and halogens.

10. The method according to claim 8, wherein the diseases associated with mitochondrial dysfunction are selected from mitochondrial disorders and neurodegenerative diseases.

11. A method for preventing mitochondrial dysfunction associated with CHCHD10 mutations, comprising the step of administering to a patient in need thereof a therapeutically effective amount of a compound of Formula I 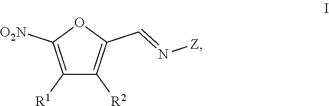
wherein
R¹ and R² are independently selected from H, C1-C6-alkyl, C3-C6-cycloalkyl and halogens, and
Z is selected from 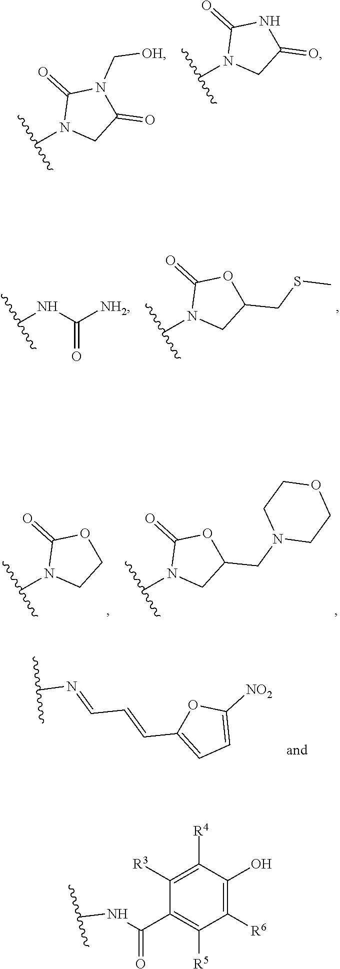
wherein R³, R⁴, R⁵ and R⁶ are independently selected from H, C1-C6-alkyl, C3-C6-cycloalkyl, OH and halogens,
or a pharmaceutically acceptable salt or solvate thereof.

12. A compound of Formula I 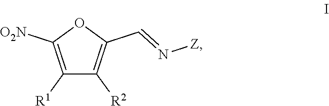
a pharmaceutically acceptable salt or a solvate thereof,
wherein
R¹ and R² are independently selected from H, C1-C6-alkyl, C3-C6-cycloalkyl and halogens, and
Z is selected from 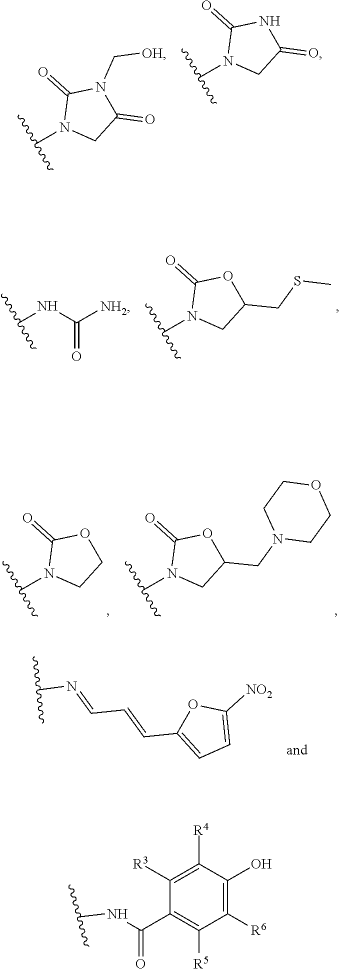
wherein R³, R⁴, R⁵ and R⁶ are independently selected from H, C1-C6-alkyl, C3-C6-cycloalkyl, OH and halogens,
with the proviso that R¹ and R² are not both H and that R³, R⁴, R⁵ and R⁶ are not all H.
